(19) United States
(12) Patent Application Publication
(10) Pub. No.: US 2017/0251674 A1
(43) Pub. Date: Sep. 7, 2017

(54) APPLICATION OF POROUS MATERIALS FOR BACTERIAL QUORUM SENSING INHIBITION/DISRUPTION

(71) Applicant: Oil-Dri Corporation of America, Chicago, IL (US)

(72) Inventors: Sajo P. Naik, Chicago, IL (US); San Ching, Chicago, IL (US); Jonathon Scholin, Chicago, IL (US); Marc A. Herpfer, Chicago, IL (US); Fang Chi, Chicago, IL (US)

(21) Appl. No.: 15/446,463

(22) Filed: Mar. 1, 2017

Related U.S. Application Data
(60) Provisional application No. 62/302,647, filed on Mar. 2, 2016.
(60) Provisional application No. 62/351,378, filed on Jun. 17, 2016.

(51) Int. Cl. A01N 59/06; A01N 63/00; A61K 38/47; A61K 33/12

(52) U.S. Cl. CPC A01N 59/06; A61K 33/12; A01N 63/00; C12Y 302/01017; A61K 38/47

(57) ABSTRACT

This application relates to the modulation of the flora of bacteria in an environment by inhibiting the quorum sensing of a specific bacteria in said environment by administering an effective amount of a quorum sensing control composition comprising at least one quorum sensing control agent, which is an adsorbent/catalytic inhibitor for a QS signal molecule, such as, N-acyl homoserine lactones (AHL), pseudomonas quinolone signal (PQS), and autoinducer-1 (AI-1), autoinducer-2 (AI-2) type of quorum sensing molecules. Quorum sensing control agents include a sorbent material, sorbent mineral or non-porous mineral such as, for example, phyllosilicate clays, silica, calcite, zeolites, diatomaceous earth, smectite, activated carbon, a nanoparticle or a combination of any of the foregoing. Methods include inhibiting the spoilage of food stuffs and preventing vibriosis in fish or shell fish.

### RELATED APPLICATIONS AND INCORPORATION BY REFERENCE

This application claims the benefit of priority to U.S. Provisional Application Ser. No. 62/302,647, filed Mar. 2, 2016 and to U.S. Provisional Application No. 62/351,378, filed Jun. 17, 2016.

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

This application relates to the modulation of the flora of bacteria in an environment by inhibiting or interfering with the quorum sensing (“QS”) of a specific targeted genus or species of bacteria in said environment by administering an effective amount of a quorum sensing control composition comprising at least one quorum sensing control agent, which is an adsorbent and/or catalytic inhibitor (hereafter “adsorbent/catalytic inhibitor”) for a QS signal molecule secreted by the targeted bacteria, such as, for example, N-acyl homoserine lactones (AHL), a pseudomonas quinolone signal (“PQS”), an autoinducer-1 (“AI-1”) signal, or an autoinducer-2 (“AI-2”) signal; this inhibition of or interference with the bacterial cell-to-cell communication is referred to as quorum quenching (“QQ”). This application further relates to quorum sensing control compositions comprising at least one quorum sensing control agent (i.e., an adsorbent/catalytic inhibitor) for a QS signal molecule, such as, for example, an AHL, a PQS, AI-1 signal, or an AI-2 signal and an inert carrier, and to methods for their preparation. Further, the present invention also relates to a method of administering an adsorbent/catalytic inhibitor for a QS signal molecule secreted from a targeted bacteria, such as, for example, an AHL, a PQS, AI-1 signal, or an AI-2 signal—to modulate quorum sensing metabolism of bacteria for better health and performance of animals or better health in humans.

### BACKGROUND OF THE INVENTION

Quorum sensing (“QS”) is a cell-to-cell communication system that allows bacteria to control features such as biofilm formation, bioluminescence, and virulence. (Miller, et al., Annu. Rev. Microbiol., 2001, 55: 165-169). Bacteria communicate using chemical signal molecules called auto inducers (“AIs”), which are produced continuously inside the bacterial cell and are subsequently secreted in the extracellular environment. When the concentration of the signal molecules reaches a threshold value, these AIs go back into the cells and regulate the gene expression to help bacteria adapt to the environmental changes. Such a regulatory system is termed as bacterial QS signal systems. QS enables unicellular bacteria to mimic multicellular organisms to accomplish certain behaviors that cannot be accomplished when they are unicellular individuals.

Quorum sensing was discovered by Nealson et al. in 1970 (K. Nealson, T. Platt, J. Hastings, J. Bacteriol. 1970; 104: 313-322) in marine bacterium V. fischeri. These researchers observed that bacteria generated bioluminescence when they reached a high population concentration. Further research using V. fischeri as a model revealed that AHLs were released by the bacteria from LuxI protein of V. fischeri. The released protein then binds with LuxR protein on the bacterial surface to modify the bacterial gene expression. Similar regulatory systems also exist in many Gram-positive or Gram-negative bacteria. When there is a modulation or shift in the population of such bacteria, there is an expression of the auto inducers synthase gene at a basic level, resulting in secretion of a small amount of autoinduction signal molecules, which diffuse extracellularly into the surrounding environment. When the bacterial population reaches a threshold value, the autoinduction signal molecules permeates into cells and will bind to transcriptional regulatory proteins to form a transcriptional regulatory protein-signal molecule polymer, which can bind to a specific DNA sequence of target genes including the synthetic gene of the signal molecule, also resulting in the production of more signal molecules. Such communication and transduction of information has been proposed in many bacteria for a long time. For example, Chromobacterium violaceum has the same mechanism as V. fischeri, and can produce C6-HSL as an auto induction molecule, the receptor protein of which is CviR.

Quorum sensing enables bacteria to coordinate behavior with surrounding and community levels through the regulation of gene expression. Specifically, activities that QS coordinates in a given population of bacteria include generation of antibiotics, bioluminescence, regulation of nitrogen-fixing gene, conjugal transfer of Ti plasmid, expression of virulent gene, pigment generation, bacterial swarming, and the formation of biofilms.) An advantage of an approach that involves interfering with the regulation of quorum sensing in bacteria is that this approach does not interfere with normal physiological functions of bacterial cells in vivo, and thus, will not prompt bacteria to develop resistance. Accordingly, administering bacterial quorum-sensing inhibitors alone or in combination with antibiotics or other antimicrobial agents susceptible to the formation of bacterial resistance, can mitigate the development of resistance or serve as an alternative to current antibiotic or antimicrobial therapy used to eliminate or treat diseases caused by bacteria.

Since the initial discovery of QS using V. fischeri by Nelson et al., a second group of bioluminescent bacteria that has a quorum sensing system regulating luminescence has been identified (C. M. Waters and B. L. Bassler, Annual Review of Cell and Developmental Biology, 2005, 21: 319-346). The aquatic bacteria V. harveyi was discovered to possess QS regulatory systems that controls the expression of various genes associated with both virulence and luminescence. The measurement of emitted luminescent light during QS signaling can, therefore, be used as an indication of the level of bacterial virulence. The bacteria V. harveyi is a common pathogen causing vibriosis, a major disease of fish and shellfish (e.g., crustaceans, mollusks etc.), resulting in serious productivity and economic losses for aquaculture industry. The production of extracellular virulence products by V. harveyi has been identified as one of the factors responsible for its pathogenesis. During cell growth, luminescence initially lags well behind growth and subsequently increases at a significantly faster rate after the autoinducers have accumulated in the medium. AI-1 signal is used for intra species communication and the AI-2 signal is used for interspecies communication. M. J. Federle and B. L. Bassler, J. Clin Invest., 2003, 112(9): 1291-1299.

Although several antibiotics have been used in controlling the population of Vibrio harveyi, such usage usually poses several problems in terms of the generation of resistance in pathogens, overuse of the drugs, and the inadvertent killing of “good” bacteria (thereby reducing the bacterial biodiversity in the environment and making it possible for pathogenic bacteria, e.g., C. difficile, to flourish). The use of antibiotics also collaterally impacts the environment; for example, the antibiotics continue to kill bacteria after the initial intended location as it enters waterways and soil and the bacteria themselves leave toxic byproducts when they are killed by the antibiotics. Thus, there are advantages of finding an alternative way of controlling the bacterial population, or the virulence and biofilm formation of them, through the administration of agents that inhibit or interfere with bacterial QS rather than using antibiotics or other drugs for mass bacterial elimination.

Diseases such as peritonitis, cholecystitis, cystitis, diarrhea, endocarditis, gastroenteritis, pyothorax, sepsis and other various diseases caused by a Gram-negative bacteria including, but not limited to E. coli, Bacillus proteus, Burkholderia cenocepacia, Pseudomonas aeruginosa, Bacillus dysenteriae, Bacillus pneumoniae, Brucella, Haemophilus influenzae, Hemophilus parainfluenzae, Moraxella catarrhalis, Acinetobacter, Yersinia, Legionella pneumophila, Bordetella pertussis, Bordetella parapertussis, Shigella spp., Pasteurella, Vibrio cholerae, and Vibrio Parahemolyticus, could be treated by administering agents that inhibit or interfere with bacterial QS alone or in combination with antibiotics or other antibacterial agents. Particularly, diseases caused by drug-resistant Gram-negative bacteria, such as C. difficile, which exhibit resistance to current antibiotic treatments, could also be treated by administering effective amounts of an agent that inhibits or interferes with bacterial QS alone or in combination with antibiotics or other antibacterial agents to treat, for example, an animal, whether it be a human, companion animal, or production animal, or a food crop, in need of such treatment.

Further, Gram-negative bacteria cause diseases in plants and these diseases could also be treated by administering agents that inhibit or interfere with bacterial QS alone or in combination with antibiotics or other antibacterial agents. Examples of Gram-negative bacteria that cause diseases in plant include, for example, Agrobacterium tumefaciens, Pantoea stewartii, Erwinia carotovora, Ralstonia solanacearum, Pseudomonas syringae, Pseudomonas aeruginosa, and Xanthomonas campestris. (S. B. von Bodman, et al., Annu. Rev. Phytopathol, 2003, 41:455-82).

QS has been reported for Gram-positive bacteria (see, M. Kleerebezem et al., Molecular Microbiology, 1997, 24(5): 895-904). Hence, it would be expected that administering agents that inhibit or interfere with bacterial QS alone or in combination with antibiotics or other antibacterial agents where the targeted bacteria are Gram-positive, would also be effective.

Bacteria can exist as a single independent cell or in the form of sessile aggregates commonly referred to as the biofilm. The shift of bacteria from single nomadic to an aggregate community occurs through quorum sensing signaling. Once formed, the aggregate community will start forming a biofilm, which about 80% of the time will cause an infection in the host, because the newly formed biofilm will turn on a virulence pathway in the bacteria. The microenvironment that attaches the bacteria to the host surface contains excreted enzymes that allow the bacteria to evade host immune responses including antibodies, intracellular pathogenesis, antigenic variation, and cellular immune responses. Such bacterial films are often difficult to treat with usual antibiotics, and as such, there is an urgent need to develop novel inhibition techniques such as through quorum sensing. Biofilms cause a significant amount of all human microbial infections, for example, in the U.S., hospital acquired (nosocomial) infections are the fourth leading cause of infections with 2 million cases annually (or 10% of American hospital patients) leading to more than $5 billion in added medical cost per annum. About 60-70% of nosocomial infections are associated with some type of implanted medical device. It is estimated that over 5 million medical devices or implants are used per annum in the U.S. alone. Microbial infections have been observed on most, if not all, such devices, including: prosthetic heart valves, orthopedic implants, intravascular catheters, artificial hearts, left ventricular assist devices, cardiac pacemakers, vascular prostheses, cerebrospinal fluid shunts, urinary catheters, ocular prostheses and contact lenses, and intrauterine contraceptive devices. (J. D. Bryers, Biotechnology and Bioengineering, 2008, 100:1; and R. P. Wenzel, Clin. Infect. Dis., 2007, 45:(Suppl 1), S85-S88.). QS signal molecules have also been associated with the spoilage of food stuffs such as vegetable and meat products (e.g., beef, lamb etc.) (V. Blana and G. Nychas, International Journal of Food Microbiology, 2014, 173: 1-8). Hence, QS disruption or quenching of QS molecules have a potential role in food processing.

AIs secreted by bacteria include following AHLs, PQS, AI-1, and AI-2 class of molecules:

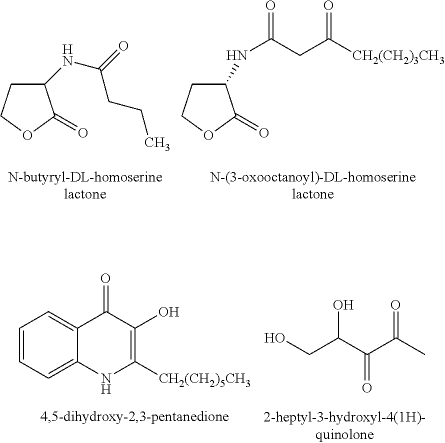

Thus, the identification or development of compounds that control or interfere with above AIs, for example, would be useful as antibacterial agents for controlling the population of unwanted bacteria and preventing the formation of biofilms.

Strategies that have been reported in the literature to disrupt bacterial QS include:
1. The inhibition of the biosynthesis of QA molecule or the enzymatic inactivation/digestion of signal molecules by the application of QS substrate analogues or enzymes using organic molecules;
2. The blockage of genetic signal expression by the application of QS antagonist again using organic molecules; and
3. The application of AS antagonists to evoke virulence factor manifestation at low population density using organic molecules.

(See, for example, T. Defoirdta, et al., Aquaculture, 2004, 240: 69-88; Y. Dong et al., Proc. Natl. Acad. Sci. U.S.A, 2000, 97:3526-3531; C. M. Waters and B. Bassler, Annu. Rev. Cell Dev. Biol., 2005, 21,319-346; B. K. Hammer and B. Bassler B., Mol. Micribiol. 2003, 50(1), 101-104). However, none of the strategies reported in the literature approaches disrupting bacterial QS signal molecules by externally adsorbing and/or deactivating QS signal molecules using a adsorbing/catalytic inhibitor of a QS signal molecule.

Compounds and methods for controlling bacteria by interfering with QS without adsorbing or deactivating QS signals are known in the art. For example, US 2012/0322769 A1 provides for compounds which control the virulence in bacteria using QS. US 2010/0256369 A1 provides for pyrimidinone compounds which have a QS inhibiting effect on specific bacteria, thereby suppressing toxin production and inhibiting the deposit of biofilms. US 2014/0275232 A1 describes analogues of 4-(4-chloro-2-methylphenoxy)-N-(2-oxotetrahydrothiophen-3-yl)butanamide of the formula:

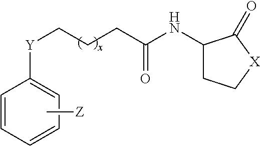

as inhibitors of quorum sensing-mediated activity in bacteria and being useful in the prevention of biofilm formations on surfaces as well as bacterial biofilm-inhibiting compositions comprising these analogues. Likewise, US 2015/0238475 A1 also provides for compounds that inhibit QS in gram-negative bacteria and teaches methods for treating an infection in a host containing said bacteria. US 2016/0002184 A1 discloses compounds of the formula:

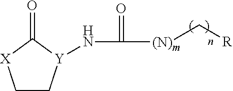

as being bacterial quorum sensing inhibitors and provides for methods for the prevention and/or treatment of a disease caused by a bacterial infection.

There have also been reports in the literature of extracts and essential oils isolated from plants and fruits of having the ability to interfere with bacterial quorum sensing. See, for example, C. L. Koh, et al., Sensors, 2014, 13:6217-6228.

The literature reports compositions comprising clays as being useful in treating diseases in animals by adsorbing toxins or in treating diseases. For example, WO 2010/028215 describes a modified fish food comprising a fish or shrimp fed material; an acidulant; and a clay material, which is reported to be effective in adsorbing aflatoxins. US 2011/0033576 describes compositions comprising yeast cell and/or yeast cell components with an altered cell wall structure (e.g., a clay or clay component interlaced into the cell wall) to sequester bacteria and toxins. US 2014/0099373 provides for methods of treating enteric disease such as those cause by Clostridium bacteria in an animal which comprises administering a mixture comprising a clay, a yeast, a yeast product or a yeast-like product to the animal. US 2016/0030475 provides for treating early mortality syndrome/acute hepatopancreatic necrosis disease in an animal in need thereof by administer a clay or a clay blend to an animal. None of these above-mentioned publications discusses using compositions comprising clays to modulate QS in bacteria.

Generally, there is a need in the art to discover new methods for combatting pathogenic bacteria. Hence, while antibacterial agents that function by interfering AIs are known, it would be advantageous to identify further compounds that are useful in controlling pathogenic bacteria in an environment, which may be used alone or in combination with, for example, known antibacterial agents.

Citation or identification of any document in this application is not an admission that such document is available as prior art to the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B and 1C illustrate the results observed using Calibrin® Z clay to disrupt QS in V. harveyi. In the experiments Calibrin® Z clay was added directly to V. harveyi bacterial culture, and bacterial luminescence and number of bacteria was monitored over time.

FIGS. 2A, 2B and 2C illustrate the results observed when four different adsorbent/catalytic inhibitors, Calibrin® Z (A), Cu-Calibrin® Z (B), H-Calibrin® Z (C) and activated carbon (D) were used to disrupt QS in V. harveyi. The adsorbent/catalytic inhibitors were added directly to V. harveyi bacterial culture, and bacterial luminescence and number of bacteria were monitored over time.

FIGS. 3A and 3B illustrate the results observed in in vitro experiments using Calibrin® Z clay to disrupt QS in V. harveyi under different conditions, where bacterial luminescence and number of bacteria were monitored over time.

FIGS. 4A and 4B illustrate the results observed when four different adsorbent/catalytic inhibitors, Calibrin® Z (A), Cu-Calibrin® Z (B), H-Calibrin® Z (C) and activated carbon (D) were used to disrupt QS in V. harveyi in in vitro experiments under different conditions, where bacterial luminescence and number of bacteria were monitored over time.

### SUMMARY OF THE INVENTION

The present invention relates to a method for modulating the flora of bacteria in an environment by inhibiting or interfering with the quorum sensing of a specific bacteria in said environment which comprises administering an effective amount of a quorum sensing control composition comprising at least one quorum sensing control agent, which is an adsorbent/catalytic inhibitor for a QS signal sensing molecule, and optionally an inert carrier to said environment.

The present invention further relates to a method for modulating the flora of bacteria in an environment by inhibiting or interfering with the quorum sensing of a specific bacteria in said environment, which comprises identifying the bacteria for which the quorum sensing is to be inhibited and administering an effective amount of a quorum sensing control composition comprising at least one quorum sensing control agent, which is an adsorbent/catalytic inhibitor of a QS signal sensing molecule, which include, for example, a AHL, PQS, AI-1 signal, or AI-2 signal, and optionally an inert carrier. Applicants have found that placing materials that exhibit adsorption/catalytic activity for AHLs, PQS, an AI-1 signal, or an AI-2 signal in an environment where the specific bacteria reside will specifically disrupt the quorum sensing of said bacteria (e.g., Clostridium sp. (for example, C. perfringens or C. difficile), Escherichia sp. (for example, E. coli), Pseudomonas sp. (for example, P. aeruginosa), Salmonella sp. (for example, S. typhimurium), or Vibrio sp. (for example, V. harveyi)), thereby reducing the negative consequences of these bacteria while not affecting the remaining bacteria.

The present invention, in an embodiment, provides for a method of modulating the flora of bacteria in an environment where the targeted bacteria are indirectly reduced as they are not stimulated to reproduce or engage in activities adverse to their host by the quorum sensing communication and continue to lead a nomadic or harmless lifestyle to the host. On the other hand, the amount of the remaining (or “good”) bacteria is either not affected or will increases due to the specificity of the added materials only against the specific AIs secreted by pathogenic bacteria. Hence, the term “modulating” in this invention refers to decreasing the population of select, undesirable bacteria in the flora or keeping the amount of select, undesirable bacteria in the flora at a level where they will continue to lead a nomadic or harmless lifestyle and keeping constant or increasing the population of desirable bacteria.

The present invention further provides for a method for inhibiting or treating unwanted bacterial growth in foodstuffs of vegetable or animal origin (e.g., beef, pigs, lamb, poultry (e.g., chickens, ducks, geese and guinea fowl, etc.) and seafood (e.g., fish and shellfish (including shrimp and other crustaceans), thereby reducing spoilage, which comprises adding an effective amount of a quorum sensing control composition comprising at least one quorum sensing control agent, which is an adsorption/catalytic inhibitor for AHLs, PQS, an AI-1 signal, or an AI-2 signal to the foodstuffs of animal or vegetable origin.

The present invention further provides for a method for preventing or treating vibriosis in fish or shellfish (e.g., shrimp) in need thereof by inhibiting the quorum sensing of Vibrio sp. (for example, V. harveyi) in an aqueous environment (e.g., aquaculture) where said fish or shellfish reside which comprises administering an effective amount of a quorum sensing control composition comprising at least one quorum sensing control agent, which is an adsorbent/catalytic inhibitor a QS signal sensing molecule, and optionally an inert carrier to said aqueous environment

This invention further relates to quorum sensing control compositions comprising and effective amount of at least one quorum sensing control agent, which comprises an adsorbent/catalytic inhibitor for a QS signal molecule, which includes, for example, AHLs, PQS, an AI-1 signal or a AI-2 signal, and an inert carrier.

This invention further provides for to a method of eliminating or reducing production of a biofilm or toxic chemicals produced by targeted genus or species of bacteria residing in an animal (including human) by eliminating or reducing a QS signal molecule, such as, for example, a AHL, a PQS, AI-1 signal, or an AI-2 signal, produced by said bacteria which comprises administering an effective amount of a quorum sensing control agent, which is an adsorbent/catalytic inhibitor for the QS signal molecule, and optionally an inert carrier.

This invention further provides for a method of eliminating or inactivating at least one QS signal molecule secreted by the targeted bacteria, such as, for example, a AHL, a PQS, AI-1 signal, or an AI-2 signal, produced by a targeted bacteria which comprises administering an effective amount of a quorum sensing control agent, which is an adsorbent/catalytic inhibitor for the QS signal molecule, and optionally an inert carrier to an environment where said targeted bacteria reside.

The choice of the adsorbent/catalytic inhibitor in any of the embodiments of the invention such as those defined above is depending on the AI to be targeted. Moreover, not only should the inhibitor be effective, it also should be safe (i.e., not toxic) to or in the environment where it is being administered. Materials that act as an adsorbent/catalytic inhibitor for a QS signal molecules, such as an AHLs, PQSs, and AI-1 signals or an AI-2 signals, by way of non-limiting examples include inorganic or organic sorptive materials, sorptive minerals, and non-porous minerals.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION OF THE INVENTION

The present invention provides for a method for modulating the flora of bacteria in an environment by inhibiting or interfering with the quorum sensing of a specific bacteria (e.g., Clostridium sp. (for example, C. perfringens or C. difficile), Escherichia sp. (for example, E. coli), Pseudomonas sp. (for example, P. aeruginosa), Salmonella sp. (for example, S. typhimurium), or Vibrio sp. (for example, V. harveyi)), in said environment, which comprises identifying the bacteria for which the quorum sensing is to be inhibited or interfered with and administering an effective amount of a quorum sensing control composition comprising at least one quorum sensing control agent, which is an adsorbent/catalytic inhibitor for the QS signal molecule from said bacteria, and optionally an inert carrier, to the environment where said targeted bacteria reside.

Another embodiment of the present invention is a method for modulating the flora of bacteria in an environment by inhibiting or interfering with the quorum sensing of a specific bacteria in said environment, wherein the environment is in or on an animal (including a human), for example the gastrointestinal tract or gut, or a foodstuff or packaging material for a foodstuff. In another embodiment, the environment is an aqueous environment or the environment is plants or soil. Non-limiting examples of animals include poultry (e.g., chickens), pigs, cattle, sheep, and companion animals (e.g., dogs, cats, birds, and rabbits).

While not wishing to be bound by theory the adsorbent/catalytic inhibitors used in the inventive methods inhibit or interfere with the QS of a targeted genus or species of bacteria by externally adsorbing and/or deactivating the QS signals molecules emitted by the targeted bacteria, thereby interrupting the cell-to-cell communication between the individual bacterium of the targeted bacteria without interfering with the internal regulation or genetic expression of QS molecule synthesis in a targeted bacterium. Hence, any compound, material or composition that externally inhibits or interferes with the cell-to-cell communication by adsorbing and/or chemically deactivating the QS signals of targeted bacteria may be used as an adsorbent/catalytic inhibitor in this invention.

### Adsorbent/Catalytic Inhibitors for QS Signal Molecules

Materials that can act as an adsorbent/catalytic inhibitor for a QS signal molecules, such as an AHLs, PQSs, and AI-1 signals or an AI-2 signals, in any of the embodiments above are by way of non-limiting examples may be inorganic compounds or materials, organic compounds or materials, or combinations thereof.

The QS adsorbent/catalytic inhibitors include clays, minerals, biopolymers or other food and non-grade materials sourced from nature/earth. However, such materials must be mined/collected, processed physically and/or chemically in order to impart functional activity to them. The QS adsorbent/catalytic inhibitors are also synthetic or commercial inorganic, organic, and organic-inorganic hybrid materials. These materials could be both regulated food-grade or otherwise. Moreover, these materials may be specifically selected because they possess an intrinsic functional activity because of a chemical or physical property or because of a functional activity that has been introduced by means of a chemical treatment, surface modification, thermal processing, ion-exchanging, vapor deposition, or by some other method, all of which would have been well known to one skilled in this art.

The selection of an adsorbent/catalytic inhibitor the control of QS in specific bacteria for a given environment will depend upon the morphology and the chemical and physical properties of the adsorbent/catalytic inhibitor. For general administration, such as, for example, to an animal or plant, the adsorbent/catalytic inhibitor should be non-toxic. Moreover, the materials should not substantially interfere with other cellular/biological functions of the living organism, plant, animal (including human), wherein such therapy is being applied. Advantageously, the particle size may be from about 1 nm to about 500 nm, or from about 10 nm to about 400 nm, about 50 nm to about 250 nm, with pore volume of about 0.1 to about 2 cm³/g, or about from about 1 cm³/g to about 1.75 cm³/g, or about 0.50 cm³ to about 0.75 cm³/g, and surface acidity of 0.01 mmol/g or 1 mmol/g or from about 0.1 mmol/g to about 0.5 mmol/g or from about 0.2 mmol/g to about 0.75 mmol/g. Some non-limiting examples of potential adsorbent/catalytic inhibitor for such application include, zeolites, clays, silica, mesoporous silica, peptide, functionalized cellulose, chitin and other biopolymers, preferably in nanoparticulate form.

One embodiment of the invention involves in vivo intravenous applications. This embodiment may require the use of non-toxic minerals, inorganic nanostructured materials, and biopolymers. Advantageously, the particle size for materials for in vivo intravenous application may be from about 1 μm to about 500 μm, or about 10 μm to about 400 μm, about 50 μm to about 250 μm, with pore volume of about 0.1 to about 1 cm³/g, or about 0.50 cm³ to about 0.75 cm³/g, and surface acidity of 0.01 mmol/g or 1 mmol/g g or from about 0.1 mmol/g to about 0.5 mmol/g or from about 0.2 mmol/g to about 0.75 mmol/g. Some non-limiting examples of potential adsorbent/catalytic inhibitor for such application include, nanoparticulate zeolites, clays, silica, mesoporous silica, peptide, functionalized cellulose, chitin and other biopolymers.

In another embodiment of the invention, the environment is external such as in foodstuff such as meat or vegetables or fruits. In these embodiments, the adsorbent/catalytic inhibitor material may be processed and drawn in the form of thin sheets, spherical granules or coated or incorporated into the container (exterior or interior) holding/carrying such food and other edible items. Non-limiting examples of adsorbent/catalytic inhibitors for these embodiments include processed and molded clays, zeolites, activated carbon, silica, mesoporous silica, and hybrid materials, such as clay nanosheets incorporated with polymers such as polyglycolide, Nafion®, polyamides, silane, chitin, dextrin, fatty acid polymer and cellulose.

### Inorganic Compounds or Materials

Inorganic compounds or materials that can act as an adsorbent/catalytic inhibitor of a QS signal molecule include inorganic compounds or materials that adsorb and/or deactivate QS signal molecules secreted from the targeted bacteria, thereby inhibiting or disrupting the cell-to-cell communication between the targeted bacteria. These compounds or materials may be porous and can capture and adsorb the QS signal molecules, where they may be held, deactivated, or both held and deactivated. Alternatively, the inorganic materials may be slightly porous or not porous and can chemically deactivate the QS signal molecule. For compounds or materials that have both adsorbent and catalytic activity, it is generally advantageous for the inorganic compounds or materials to have BET surface areas in excess of about 100 m²/g for clays (EGME surface area above about 500 m²/g), high mesopore volume in excess of about 0.2 cm³ and acidity in terms of TPD-NH₃, equivalent to about 0.010 mmol/g or 1 mmol/g or from about 0.1 mmol/g to about 0.5 mmol/g or from about 0.2 mmol/g to about 0.75 mmol/g are preferred. Measuring acidity in terms of TDP-NH₃ is a standard method that is recognized in this art (see, I. M. Sawalha, et al., Journal of Chemical, Molecular, Nuclear, Materials and Metallurgical Engineering, 2011, 5(7), 570-574: For compounds or materials with adsorbent activity. It is generally advantageous for the compounds or materials to exhibit high surface area together with very low acidity. Non-limiting examples of inorganic compounds or materials include sorptive minerals, sorbent minerals, inorganic sorptive materials (e.g., porous nanoparticles), synthetic zeolites, mesoporous silica, pure and laboratory functionalized diatomaceous earth, or combinations thereof.

### 1. Sorptive Minerals

Sorptive minerals are minerals that both adsorbs and deactivates a QS signal molecule. Illustrative examples include clay minerals and clays (clay minerals with traces of metal oxides and organic matter), and sorbent minerals.

a. Clay Minerals

Clay minerals are hydrous aluminum phyllosilicates, which may contain variable amounts of iron, magnesium, alkali metals, alkaline earths and other cations. Clay minerals exist in nature but have to be further processed for them to possess the chemical or physical properties necessary for them to be useful. This processing may include both physical and chemical treatments. Clay directly obtained from earth may contain a multitude of other non-clay minerals, (e.g., top soil, quartz, silica, etc.) associated with it. However, crushing, sieving (about 20 to about 400 mesh size), sizing (about 1 to about 100 μm particle size or from about 20 to about 50 μm), thermal processing (about 100 to about 800° C.), wet processing, chemical treatment, ion-exchanging, functionalization, and such treatment will impart desired properties to the clay mineral that will impart specific properties that lead to toxin binding, catalysis, adsorption, etc.

b. Clays

The clays used in this invention are processed clays that have been mechanically processed and optionally chemically or thermally treated; chemical treatments involve, for example, reacting the clay with an acid, base (e.g. alkali) or salt solution. In one embodiment the processed clays thermally processed clays that are the advantageously heated to a temperature between about 100 to about 800° C. (for example, about 400 to about 800° C.) and ground to a fine particle size (e.g., to a particle size of approximately between about 10 microns to as large as about 500 microns or advantageously between about 20 and about 50 microns) (“heat treated clays”). Methods to make processed clays are well known to a person of ordinary skill in this art. Non-limiting examples of clays which may be processed are: clay minerals, such as smectites (which include montmorillonite, nontronite, beidellite and saponite); alumino-silicate, sepiolite, phyllosilicates; attapulgite (palygorskite); bentonite (e.g., sodium bentonite); hormite, kaolin; and fuller's earth.

In some embodiments the clay may be heated to about 100° C., about 125° C., about 150° C., about 175° C., about 200° C., about 225° C., about 250° C., about 275° C., about 300° C., about 325° C., about 350° C., about 375° C., about 400° C., about 425° C., about 450° C., about 475° C., about 500° C., about 525° C., about 550° C., about 575° C., about 600° C., about 625° C., about 650° C., about 675° C., about 700° C., about 725° C., about 750° C., about 775° C., about 800° C., about 825° C., about 850° C., about 875° C., about 900° C., about 925° C., about 950° C., or about 1000° C. It may be heated for 1 minute up to 12 hours or between about 1 to about 4 hours. Heating may be done statically in a muffled furnace or dynamically in a flash dryer.

In some embodiments of the present invention the processed clays are montmorillonite clay, attapulgite clay, or hormite, or sodium bentonite, which have been heat treated at a temperature between about 100 to about 800° C.

Non-limiting examples of processed clays are heat treated clays, such as heat treated montmorillonite clays, which have been heat treated at a temperature of between about 100° C. to about 800° C. and have an average particle size between about 32 microns to about 36 microns, such as, for example, Calibrin®-A, Calibrin®-TQ or Calibrin®-Z.

In some embodiments the present invention uses ion-exchanged or functionalized clays. An “ion-exchanged clay” is a processed clay, such as one of those identified above, that has been reacted with an ion-exchange material. Processes to prepare ion-exchanged clays are well known to one of ordinary skill in this art (for example, D. Carrol, Geological Society of America, 1959, 70(6): 749-779) and processes to prepare these clays are described in more detail below. Generally, the clay is dispersed and stirred aggressively in a salt solution, which contains the cation to be exchanged (e.g. CuC1₂), at a fixed temperature for a fixed amount time. During this process, the naturally present cations in the clay interlayer exude out of the structure and the cations from the salt solution (Cu²⁺) take their place in the clay structure. The clay thus formed may impart different properties than the parent clay due to the presence of different cations (e.g., copper ions) in its structure.

Non-limiting examples of ion-exchanged clays include aluminum, copper or proton exchanged montmorillonite clay; e.g., H-montmorillonite, Al-montmorillonite and Cu-montmorillonite. Non-limiting examples of these clays include copper exchanged Calibrin®-A or copper exchanged Calibrin®-Z, aluminum exchanged Calibrin®-A or aluminum exchanged Calibrin®-Z, or proton exchanged Calibrin®-Z.

A “functionalized clay” is a processed clay in which chemical functionalities or an active and specific organic group has been added to the surface of the clay to enhance specific properties of the processed clay or hybrid. Hybrid refers to the formation of a new material containing both inorganic and organic functionalities and are also called hybrid materials. Hybrid materials can exhibit both inorganic and organic properties; e.g., a polymer infused clay is a hybrid which will exhibit the flexibility of a polymer (organic property) and the strength of a clay (inorganic property). A functionalized clay is obtained by reacting a modified clay, such as those heat treated clays identified above, with an amino acid (e.g., histidine or isoleucine), protein (e.g., lysozyme, peptides, etc.). Processes to functionalize clays are well known to one of ordinary skill in the art and processes to prepare these clays are described below.

Non-limiting examples include Calibrin®-A-histidine, Calibrin®-A-isoleucine, Calibrin®-A-lysozyme, or attapulgite-lysozyme.

In some embodiments, the processed clay in ion-exchanged modified clay or a functionalized modified clay is heat treated montmorillonite clay, attapulgite clay, or hormite, or sodium bentonite.

In the above discussion “montmorillonite clay” refers to a clay which is at least 50% montmorillonite, such as the clay found in the Porter's Creek Formation, which is mined in Mississippi, Illinois, Missouri, and Tennessee. Clay minerals are fundamentally constructed of tetrahedral silicate sheets and octahedral hydroxide sheets and are classified as 1:1 or 2:1 clays. A 1:1 clay consists of one tetrahedral sheet and one octahedral sheet, e.g. kaolinite. A 2:1 clay consists of an octahedral sheet sandwiched between two tetrahedral sheets, e.g. montmorillonite. The smectite group includes dioctahedral smectites (e.g. montmorillonite, nontronite and beidellite) and trioctahedral smectites (e.g. saponite). The illite group includes the clay-micas. Other 2:1 clay types which exist include clays such as sepiolite or attapulgite; these clays have long water channels internal to their structure.

c. Sorbent Minerals

Sorbent minerals are minerals that can adsorb or absorb solids, liquids or gases; sorbent minerals only catalytically deactivate the QS molecules under certain conditions. Illustrative examples include, zeolites, silica, calcite, illite, volcanic silica, mica, and perlite and combinations of these materials. These materials are mechanically processed and optionally thermally or chemically treated. These processes involve increasing or decreasing the drying temperature, time or final moisture content or calcining the material or treating the minerals with a salt solution under heat to have a thermal ion exchange.

The sorbent minerals may be ground to a fine particle size (e.g., to a particle size of approximately between about 1 μm to about 500 μm, more advantageously from about 10 μm-about 400 μm, about 50 μm to about 250 μm or about between 20 and 50 microns. Moreover, the sorbent minerals may be advantageously heated to a temperature between 100-800° C. (for example about 400 to about 800° C.).

In some embodiments the sorbent mineral may be advantageously heated to about 100° C., about 125° C., about 150° C., about 175° C., about 200° C., about 225° C., about 250° C., about 275° C., about 300° C., about 325° C., about 350° C., about 375° C., about 400° C., about 425° C., about 450° C., about 475° C., about 500° C., about 525° C., about 550° C., about 575° C., about 600° C., about 625° C., about 650° C., about 675° C., about 700° C., about 725° C., about 750° C., about 775° C., about 800° C., about 825° C., about 850° C., about 875° C., about 900° C., about 925° C., about 950° C., or about 1000° C. It may be heated for 1 minute up to 24 hours or between about 1 to about 4 hours.

In one of embodiment of this invention the sorbent mineral is (e.g., HY-zeolite), or tetrasilicate.

### 2. Non Porous Minerals or Materials

“Non-porous mineral” is a mineral that deactivates the QS signal molecule by catalytic degradation of the molecule only; it does not adsorb the QS signal molecule or only adsorbs the QS signal molecule to a limited extent. Non-porous materials function in the same way as non-porous minerals and possess a pore volume which is close to zero. The BET surface area of such large non-porous minerals would be in the range of 2 to 10 m²/g. The particle size of such materials varies from about 2 μm to about 500 μm. Non-limiting examples of non-porous minerals include aluminum oxides, silica oxides, iron oxides, AlCl₃, copper oxides, and calcium oxides. Non-limiting examples of non-porous materials include micrometer-sized ZnO, MgO, Al₂O₃—SiO₂, TiO₂, etc. These compounds are commercially available, and can be modified by dilute acid or base wash, and grinding to enhance performance.

In one embodiment, the non-porous mineral is AlCl₃, copper oxide, acid functionalized non-porous silica, layered oxides and hydroxides (e.g., M-Al hydrotalcites).

In another embodiment the non-porous mineral is oxide of aluminum or aluminum chloride.

### 3. Nanoparticles

Nanoparticles are siliceous, aluminosilicates or oxides. They include colloidal silica, colloidal zeolites, precipitated and fumed silica. The particle size very from about 5 nm to about 100 nm, and possess a surface area between about 50 to about 500 m²/g. Nanoparticles are created or sourced for this application to replicate the functionality of processed clays or non-porous materials in either deactivating the QS signal molecule by catalytic degradation, adsorption, or a combination thereof. These materials are commercially available.

### Organic Compounds or Materials

Organic compounds or materials that can act as adsorbent/catalytic inhibitors materials include sorptive organic compounds, sorptive natural products, sorptive articles of manufacture, or mixtures thereof; these organic compounds are porous on their surfaces and can adsorb and/or can deactivate a QS molecule. Non-limiting examples of sorptive organic compounds or sorptive organic materials include synthetic materials isolated from biomass, such as activated carbon, which is porous and has a surface area of about 1200 m²/g, and pore volume of about 0.4 cm³/g, woody biomass, or humic acid; non-porous biopolymers, such as polyamide and polyglycolide; porous biopolymers (e.g., chitosan, cellulose, dextrin, polysaccharides, lignin, proteins, fatty acid polymer, and peptides); synthetic porous polymers, such as sulfonated tetrafluoroethylene based fluoropolymer-copolymer (Nafion®), which may be further functionalized; and synthetic non-porous polymers such as optionally functionalized poly(vinylpyridines) and polyacrylates. These compounds or materials are commercially available or are readily synthesized from procedures well known to one skilled in this art.

### Environment

This invention contemplates using the inventive methods wherever the targeted bacteria reside. The environments may be in vitro, i.e., places outside living organisms or in vivo, i.e., places inside a living organism.

### In Vitro Environments

In vitro environments include external surface areas where the targeted bacteria congregate, such as household fixtures, countertops, surgical instruments, food processing equipment, food packaging equipment, food packaging, food products, including agricultural products, such as seeds, fruits and vegetables, or processed foods. For agricultural products, the environment might be on the seeds, fruits or vegetables, on the crops plants or in the field (including the soil) where the crops or plants are being cultivated. Similarly, the environment may be processed foods or places where such foods are processed. Moreover, environments include places where animals are raised or reside, such as aqueous environments for raising fish or animal bedding. Other in vitro environments include drinking water for animals (including humans), activated sludge or other areas in the treatment of waste.

The quorum sensing control compositions may be solid or liquid and may be formulated as sprays.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. The adsorbent/catalytic inhibitor can be (micro)encapsulated and further formed into a suspension or solid formulation; alternatively the entire formulation can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulations.

Sprayable formulations are typically suspended in a suitable medium before spraying. Such liquid and solid formulations are formulated to be readily diluted in the spray medium, usually water. Spray volumes depend upon the environment being treated and the determination of the spray volume is well within the skill level of one of ordinary skill in the art.

For example, in agriculture applications, the spray volumes can range from about from about one to several thousand liters per hectare, but more typically are in the range from about ten to several hundred liters per hectare. When the sprayable formulations are for agriculture application, the formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic update.

The quorum sensing control compositions will typically contain effective amounts of the adsorbent/catalytic control inhibitor of between about 5 to 95% (w/w); between about 35 to 75% (w/w); or about 50 to 90% (w/w) based upon the total weight of the formulation. Additional formulation adjuvants include inert diluents or carriers and surfactants.

Solid diluents are well known to one of ordinary skill in this art and can include, for example, gypsum, titanium dioxide, zinc oxide, starch, sugars (e.g., lactose, sucrose) urea, calcium carbonate, sodium carbonate and bicarbonate, and sodium sulfate.

Liquid diluents include, include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyl PEG (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1997.

The quorum sensing control compositions may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.)

As mentioned above, one embodiment of the methods according to this invention is by spraying. Alternatively, a granular composition comprising an adsorbent/catalytic inhibitor of the invention can be applied to the plant foliage, the soil or some other surface where the targeted bacteria reside.

In one embodiment of the present invention the crop is potatoes and the targeted bacteria are those that effect potatoes, such as Pectobacterium atrosepticum (Pba) and Pectobacterium carotovorum, which cause blackleg (stem rotting) and soft rot. Hence, one embodiment of this invention provides for a method of preventing or treating bacterial diseases that affect potatoes, such as blackleg or soft rot, by applying an effective amount of a quorum sensing control composition according to this invention to the potato plants or to an environment where the potato plans reside (e.g., the field or the soil).

The quorum sensing control agents or compositions of this invention are also effective by localized application to the locus where the targeted bacteria reside. Methods of contact include application of a compound or a composition of the invention by direct and residual sprays, aerial sprays, gels, seed coatings, microencapsulations, systemic uptake, boluses, aerosols, dusts and many others. The quorum sensing control agents of this invention may also be applied to external surfaces, such a countertops or surgical instruments or food processing equipment, or impregnated into materials for fabricating bacterial control devices; this might include animal bedding.

In one embodiment the formulations for the quorum sensing control compositions may be added to an environment where an animal resides. In a non-limiting example, a quorum sensing control composition of the present invention is added to an aqueous environment where fish or shellfish reside. In one embodiment the amount of adsorbent/catalytic inhibitor is from about 1% to about 90% (wt/wt); about 1% to about 75% (wt/wt); about 1% to about 50% (wt/wt); about 1% to about 25% (wt/wt); or an amount within these ranges based upon total weight of the formulation. Dosages range from about 0.05 to about 5000 mg/kg of body weight/day more preferably from about 100 to about 1000 mg/kg/day. Diluents and carriers include those listed above which are approved for aquaculture use.

Advantageously in one embodiment of the present invention the quorum sensing control agents or compositions do not contain at least one of an acidulant, such as acidic calcium sulfate, yeast, a yeast component, a yeast fermentation product, yeast mannans, a yeast comprising an altered cell wall structure, or an immuno modulator, such as for example, a glutamic acid, α-ketoglutarate, glutamine, L-glutamic acid or L-glutamine or a derivative thereof. In another embodiment the QS control compositions do not contain an active agent other than the adsorbent/catalytic inhibitors of the present invention. In yet another embodiment, the QS control compositions do not contain active agents other than the adsorbent/catalytic inhibitors of the present invention and at least one antibiotic.

Suitable intervals for the administration of adsorbent/catalytic inhibitors of the present invention to range from about daily to about yearly. Of note are administration intervals ranging from daily or weekly to about once every 6 months. Of particular note are monthly administration intervals. In another embodiment, the quorum sensing control compositions for aquaculture are applied for a period of up 30 days, with some embodiments being 5, 10, or 15 days.

In another embodiment, the quorum sensing control compositions may be applied to foodstuffs to prevent spoilage by the targeted bacteria. On one embodiment, the amount of adsorbent/catalytic inhibitor is form about 0.001 to about 10 weight % Diluents and carriers include those listed above which are approved for use in food stuffs. Suitable intervals for the administration of the adsorbent/catalytic inhibitor include every second, third, fourth, fifth, sixth, seventh, eighth or nine day or some time interval in between.

The formulations for quorum sensing control compositions may be used in protecting seeds from the targeted bacteria. In the context of the present invention the seed is contacted with an effective amount of a quorum sensing control composition.

The frequency of applying the quorum sensing control compositions to the environment depends upon the nature of the in vivo environment and it is well within the skill level of one of ordinary skill in the art to determine the frequency of applying the quorum sensing control compositions for a particular environment. In one embodiment the quorum sensing control compositions may be applied just once. In other embodiments the quorum sensing control composition might be applied once or twice a day for a period of time, such as for example, 2, 3, 5, 10 or 15 days or some time period in between.

In another preferred embodiment, the QS control compositions of the invention may be formulated for addition to the drinking water of animals (including humans). The amount of adsorbent/catalytic inhibitor is from about 5 to about 100 mg/kg of body weight/day more preferably from about 5 mg/kg/day.

When the QS controlled composition is an algaecide, the inert carrier includes carriers such as water, polymer suspensions, gels and sols. When used as an algaecide, the amount added to the aqueous environment is about 0.01 to about 50%. Methods to making algaecides are well known to one of ordinary skill in this art.

### In Vivo Environments

In vitro environments include areas or places on or inside a living organism, such as an animal (including humans) where the targeted bacteria reside. Animals include, cattle, pigs, lamb, birds (e.g., chickens, ducks, geese and guinea fowl etc.), horses, camels, deer, donkeys, buffaloes, antelopes, rabbits, companion animals (e.g., dogs, cats, rabbits, etc.), rodents, turtles, fish and shellfish (including shrimp and other crustaceans). Areas or places on or inside include, for example, skin surface of a human or animal or is the gastrointestinal tract, nasal passages, urinal tract, vaginal tract, or gut of a human or animal.

The quorum sensing control compositions may be solid or liquid. Typically the formulations contain acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content.

Thus, the quorum sensing control compositions for human or animal administration may take the form of any pharmaceutically or veterinarally dosage form that would be known to one of ordinary skill in this art; these include controlled-release dosage forms. Solid forms for oral or rectal administration may contain pharmaceutically or veterinarally acceptable binders, sweeteners, disintegrating agents, diluents, flavorings, coating agents, preservatives, lubricants and/or time delay agents. Suitable binders include gum acacia, gelatin, corn starch, gum tragacanth, sodium alginate, carboxymethylcellulose or polyethylene glycol. Suitable sweeteners include sucrose, lactose, glucose or flavonoid glycosides such as neohesperidine dihydrochalcone. Suitable disintegrating agents include corn starch, methylcellulose, polyvinlypyrrolidone, xanthan gum, alginic acid or agar. Suitable diluents include lactose, sorbitol, mannitol, dextrose, cellulose, calcium carbonate, calcium silicate or dicalcium phosphate. Suitable flavoring agents include peppermint oil, oil of wintergreen, cherry, orange or raspberry flavorings. Suitable coating agents include polymers or copolymers of acrylic acid and/or methacrylic acid and/or their esters, and/or their amides, waxes, fatty alcohols, zein, shellac or gluten. Suitable preservatives include sodium benzoate, vitamin E, α-tocopherol, ascorbic acid, methyl parabens, propyl parabens or sodium bisulphate. Suitable lubricants include magnesium stearate, stearic acid, sodium oleate, sodium chloride or talc. Suitable time delay agents for controlled release formulations, include glyceryl monostearate or glyceryl distearate.

Suspensions for oral or rectal administration may further comprise dispersing agents and/or suspending agents. Suitable suspending agents include sodium carboxylmethylcellulose, methylcellulose, hydroxypropylmethylcellulose, polyvinylpyrrolidone, sodium alginate or cetyl alcohol. Suitable dispersing agents include lecithin, polyoxyethylene esters or fatty acids such as stearic acid, polyoxyethylene sorbitol mono- or di-oleate, -stearate or -laurate, polyoxyethylene sorbitan mono- or di-oleate, -stearate or -laurate and the like.

For parenteral administration, including intravenous, intramuscular and subcutaneous injection, a compound of the present invention can be formulated in suspension, solution or emulsion in oily or aqueous vehicles, and may contain adjuncts such as suspending, stabilizing and/or dispersing agents. The adsorbent/catalytic inhibitors of the present invention may also be formulated for bolus injection or continuous infusion. Pharmaceutical compositions for injection include aqueous solutions preferably in physiologically compatible buffers containing other excipients or auxiliaries as are known in the art of pharmaceutical formulation. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Formulations for injection may be presented in unit dosage form, e.g., in ampoules or in multi-dose containers. Alternatively, the active ingredient may be in powder form for constitution with a suitable vehicle, e.g., sterile, pyrogen-free water before use.

Formulations for acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content.

A pour-on formulation may also be prepared for control of parasites in an animal of agricultural value. The pour-on formulations of this invention can be in the form of a liquid, powder, emulsion, foam, paste, aerosol, ointment, salve or gel. Typically, the pour-on formulation is liquid. These pour-on formulations can be effectively applied to sheep, cattle, goats, other ruminants, camelids, pigs and horses. The pour-on formulation is typically applied by pouring in one or several lines or in a spot-on the dorsal midline (back) or shoulder of an animal. More typically, the formulation is applied by pouring it along the back of the animal, following the spine. The formulation can also be applied to the animal by other conventional methods, including wiping an impregnated material over at least a small area of the animal, or applying it using a commercially available applicator, by means of a syringe, by spraying or by using a spray race. The pour-on formulations include a carrier and can also include one or more additional ingredients. Examples of suitable additional ingredients are stabilizers such as antioxidants, spreading agents, preservatives, adhesion promoters, active solubilisers such as oleic acid, viscosity modifiers, UV blockers or absorbers, and colorants. Surface active agents, including anionic, cationic, non-ionic and ampholytic surface active agents, can also be included in these

The formulations of this invention typically include an antioxidant, such as BHT (butylated hydroxytoluene). The antioxidant is generally present in amounts of at about 0.1-5% (wt/wt). Some of the formulations require a solubilizer, such as oleic acid, to dissolve the active agent, particularly if spinosad is used. Common spreading agents used in these pour-on formulations include isopropyl myristate, isopropyl palmitate, caprylic/capric acid esters of saturated C₁₂-C₁₈ fatty alcohols, oleic acid, oleyl ester, ethyl oleate, triglycerides, silicone oils and dipropylene glycol methyl ether. The pour-on formulations of this invention are prepared according to known techniques. When the pour-on formulation is a solution, the adsorbent/catalytic inhibitor is mixed with the carrier or vehicle, using heat and stirring if required. Auxiliary or additional ingredients can be added to the mixture of active agent and carrier, or they can be mixed with the active agent prior to the addition of the carrier. If the pour-on formulation is an emulsion or suspension, the formulations can be similarly prepared using known techniques.

Other delivery systems for relatively hydrophobic pharmaceutical compounds can be employed. Liposomes and emulsions are well-known examples of delivery vehicles or carriers for hydrophobic drugs. In addition, organic solvents such as dimethylsulfoxide can be used, if needed.

In another embodiment, the formulation may be chewable and/or edible product (e.g., a chewable treat or edible tablet). Such a product would ideally have a taste, texture and/or aroma favored by the animal or human to be protected so as to facilitate oral administration

For oral, subcutaneous or spot-on administration to homeothermic animals, a dose of an adsorbent/catalytic inhibitor of the present invention administered at suitable intervals typically ranges from about 0.01 mg/kg to about 100 mg/kg, and preferably from about 0.01 mg/kg to about 30 mg/kg of animal body weight. For other topical (e.g., dermal) administration, including dips and sprays, a dose typically contains from about 0.01 ppm to about 150,000 ppm, more typically from about 0.01 ppm to about 100,000 ppm, preferably from about 0.01 ppm to about 5,000 ppm, and most preferably from about 0.01 ppm to about 3,000 ppm, of an adsorbent/catalytic inhibitor of a present invention.

Suitable intervals for the administration of compounds of the present invention to homeothermic animals range from about daily to about yearly. Of note are administration intervals ranging from about weekly to about once every 6 months. Of particular note are monthly administration intervals (i.e. administering the compound to the animal once every month).

The quorum sensing control formulations of the present invention may also include one or more antibiotics. Useful antibiotic include fluoroquinolones, such as, for example, enrofloxacin, danofloxacin, difloxacin, orbifloxacin and marbofloxacin. In the case of enrofloxacin, it may be administered in a concentration of about 100 mg/mL. Danofloxacin may be present in a concentration of about 180 mg/ml. Other useful antibiotics include tetracyclines, particularly chlortetracycline and oxytetracycline. Other antibiotics may include β-lactams such as penicillins, e.g., penicillin, ampicillin, amoxicillin, or a combination of amoxicillin with clavulanic acid or other beta lactamase.

In other embodiments, where the in vivo environment is an animal (including human), suitable diluents or carriers include

The choice of inert carriers to be included in the quorum sensing composition depends upon the environment. When the environment is an animal (including a human), suitable inert carriers for the QS control composition include water, vegetable oils (e.g., olive oil, peanut or arachis oil, sesame oil, rapeseed oil, palm oil, soybean oil, sunflower oil, safflower oil, or coconut oil), essential oils (e.g., anise oil calamus oil, or cinnamon, oil), aliphatic, aromatic, saturate or unsaturated free fatty acids and their derivatives, liquid paraffin, ethylene glycol, propylene glycol, polyethylene glycol, ethanol, propanol, isopropanol, glycerol, fatty alcohols, triglycerides, polyvinyl alcohol, partially hydrolyzed polyvinyl acetate and mixtures thereof.

In some embodiment of the invention, for oral administration, the pharmaceutical or veterinary composition may be in the form of tablets, lozenges, pills, troches, capsules, elixirs, powders, including lyophilized powders, solutions, granules, suspensions, emulsions, syrups and tinctures. Slow-release, or delayed-release, forms may also be prepared, for example in the form of coated particles, multi-layer tablets or microgranules.

The invention also provides an animal feed composition comprising the QS control compositions of the invention and a feedstuff. The quorum sensing control compositions is preferably present in an amount of from about 0.01 to about 10% of the total feed composition and preferably from 0.1 to 5% of the total feed composition, more preferably about 1% of the total feed composition.

Generally, the QS control compositions for administration in the method of the invention may be prepared by means known in the art for the preparation of compositions (such as in the art of veterinary and pharmaceutical compositions) including blending, grinding, homogenizing, suspending, dissolving, emulsifying, dispersing and where appropriate, mixing of the components together with selected excipients, diluents, carriers and adjuvants.

The term “effective amount” as used herein means that amount of a quorum sensing control agent which disrupts the quorum sensing ability of the bacteria in question. Typically, disruption occurs when the amount of the quorum sensing control agent exceeds the energy barrier for adsorption or catalysis process. The exact nature of disruption (i.e. by adsorption or catalysis) is quantified by HPLC/LC-MS analysis of the liquid extract. The quantification of catalysis and adsorption is performed from the quantitative analytical data that identifies individual reaction products and their computation to estimate their weight percentages. Exemplary ranges for the amounts of quorum sensing control agents present in the quorum sensing control compositions are between about 1 and about 50,000 weight ratio of quorum sensing control agent to quorum sensing molecule.

An “inert carrier” is an inorganic or organic material that does not react with the other components in the quorum sensing composition or with the active components loaded onto it. An “inert carrier” may react with components that are not in the quorum sensing composition.

### Processes to Prepare Ion-Exchanged and Functionalized Clays

The following provides some general methods used to prepare functionalized clays.

### Procedure to Produce Amino Acid Functionalized Clay

Amino acid modified clays are prepared by mixing a fixed amount of a clay, such as montmorillonite, into a 1000 ppm solution of an amino acid, such as L-isoleucine or L-histidine and centrifuged, for example, for approximately 30 minutes at about 400 rpm. The solutions are then centrifuged, for example at about 3,500 rpm for 30 minutes, to recover the functionalized clay. The recovered functionalized clay is then successively washed with 500 ml of deionized water to remove any loosely bound amino acids.

### Procedure to Prepare Al-Montmorillonite, H-Montmorillonite, Cu-Montmorillonite

Montmorillonite was ground and washed in deionized water at a ratio of 10 g clay:100 ml water for 24 h under agitation. The resulting clay suspension was centrifuged and the wash water discarded. The clay was rehydrated with 100 ml water to which the source for Al³⁺, Cu²⁺, H⁺ cations (e.g., CuSO₄.5H₂O, Al₂(SO₄)₃, HCl, etc.) was added at an amount of 2 times the CEC of the clay. The resulting slurry was then agitated at 40° C. for 24 h. The ion-exchanged clay was then separated by centrifugation and washed until free from the anions. The washed material was dried at 105° C., 12 h, and then ground in an agate mortar.

### Procedure to Prepare Purified Montmorillonite

A 500 g of raw montmorillonite clay was dispersed in 5 L of deionized water with aggressive stirring using an overhead stirrer. The slurry was passed through a size 350 test sieve (45 μm) by gently rubbing the finger against the screen. The sol was collected and centrifuged at 3000 rpm for 1 h. The supernatant containing the dispersed clay was re-centrifuged to separate the heavier fraction once again. The supernatant was collected and centrifuged once again, and the whole process was repeated for a few more cycles until pure montmorillonite was obtained.

### Procedure to Prepare Montmorillonite Fines

Calibrin® TQ, ultrafine fraction (average size distribution of 10 micrometer particles) of montmorillonite was prepared using a proprietary alpine or air classification particle separation method.

### Preparation of Calibrin®-A-Lysozyme or Attapulgite-Lysozyme Modified Clay

Approximately 10 g of base clay material (Calibrin®-A or attapulgite) were placed in a bottle and then 100 mL of water was added. The mixture was stirred at room temperature for 30 min, to this solution was added 50 mL of lysozyme stock solution (1-10 weight %). The bottle was capped and then shaken at 250 rpm and 25° C. for 16 hours. After the shaking was completed the mixture was centrifuge at 5,000 rpm for 30 minutes to collect the lysozyme functionalized clay.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

The purpose of these examples were to evaluate the effect of different clays on three known AIs: N-(3-oxooctanoyl)-L-homoserine lactone, 2-heptyl-3-hydroxyl-4-quinolone (PQS) and 4,5-dihydroxy-2,3-pentanedione (AI-2 signal).

### Example 1 Adsorption/Catalysis of N-butyryl-DL-homoserine Lactone on Various Adsorbent/Catalytic Inhibitors

An aqueous solution of N-butyryl-DL-homoserine lactone was taken in a small vial containing a fixed amount of clay to achieve an adsorbent/catalytic inhibitor/analyte ratio of 15 (mg/mg). The suspension was agitated at 25° C. for 30 minutes followed by centrifugation at 4500 rpm for 30 minute. The supernatant was drawn off and directly analyzed with HPLC-DAD using the conditions listed in Table 1.

TABLE 1
HPLC-DAD methodology for quantification
of N-butyryl-DL-homoserine lactone.

| | |
|---|---|
| Column: | Agilent Zorbax RX-C8 column (150 mm × 4.6 mm × 5 μm) |
| Flow Rate: | 1.0 mL/min |
| DAD: | 202 nm |

| Time (minutes) | % HPLC Water | % Methanol |
|---|---|---|
| 0.0-7.0 | 30 | 70 |

The performance of different adsorbent/catalytic inhibitors for N-butyryl-DL-homoserine lactone is provided in Table 2.

TABLE 2
The performance of different adsorbent/catalytic inhibitors
for the removal N-butyryl-DL-homoserine lactone at adsorbent/catalytic
inhibitor/QS analyte ratio of 15 (mg/mg).

| Material | % QS Removal |
|---|---|
| Calibrin ®-Z | 53 |
| Calibrin ®-A-Lysozyme | 56 |
| Cu-Montmorillonite | 65 |

### Example 2 Adsorption/Catalysis of N-(3-oxoctanyol)-L-homoserine Lactone on Various Adsorbent/Catalytic Inhibitors

A 200 ppm aqueous solution of N-(3-oxooctanoyl)-DL-homoserine lactone was taken in a small vial containing a fixed amount of clay to achieve an adsorbent/catalytic inhibitor/QS analyte ratio of 375 (mg/mg). The suspension was agitated at 100 rpm for 15 minutes, and successively centrifuged at 3,500 rpm for 30 minutes. The supernatant was drawn off and directly analyzed with HPLC-DAD using the conditions listed in Table 3. Degradation and polymerization products were identified with LC/MS.

TABLE 3
HPLC-DAD methodology for quantification of
N-(3-oxooctanoyl)-DL-homoserine lactone.

| | |
|---|---|
| Column: | Agilent Zorbax RX-C8 column (150 mm × 4.6 mm × 5 μm) |
| Flow Rate: | 0.7 ml/min |
| DAD: | 210 nm |

| Time (minutes) | % Acetonitrile | % HPLC water |
|---|---|---|
| 0.0 | 50 | 50 |
| 15.0 | 100 | 0 |

The performance of different adsorbent/catalytic inhibitors for the removal of N-(3-oxooctanoyl)-DL-homoserine lactone is provided in Table 4.

TABLE 4
The performance of different adsorbent/catalytic inhibitors for
the removal of N-(3-oxooctanoyl)-DL-homoserine lactone at an
adsorbent/catalytic inhibitor/QS analyte ratio of 375 (mg/mg).

| | BET | Pore | TPD-NH_{3,} | |
|---|---|---|---|---|
| | surface | volume, | acidity | % QS |
| | area | DFT | (150-350° C.) | Re- |
| Material | (m²/g) | (cm³/g) | mmol/g | moval |
| Calibrin ®-Z | 110 | 0.26 | 0.024 | 59 |
| HY-Zeolite | 780 | 0.41 | 0.035 | 63 |
| Illite | 70 | 0.06 | 0.024 | 36 |
| Attapulgite | 180 | 0.29 | 0.025 | 21 |
| Activated Carbon | 980 | 0.56 | 0.001 | 100 |
| Purified-Calibrin-A | 132 | 0.23 | 0.028 | 62 |
| Calibrin ®-A-Lysozyme | 93 | 0.21 | — | 61 |
| Attapulgite-Lysozyme- | 160 | 0.30 | — | 54 |
| Lysozyme | | | | |
| Calibrin ®-A-Histidine | 120 | 0.28 | — | 60 |
| Calibrin ®-A-lsoleucine | 118 | 0.32 | — | 61 |
| Cu-Montmorillonite | 125 | 0.24 | 0.035 | 66 |
| Al-Montmorillonite | 132 | 0.24 | 0.038 | 74 |
| H-Montmorillonite | 145 | 0.24 | 0.042 | 80 |
| Calibrin ®-A | 110 | 0.32 | 0.025 | 53 |
| AlCl₃ | — | — | — | 19 |
| Precipitated SiO₂ | 340 | 1.00 | 0 | 25 |
| Calibrin ®-Z Fines | | 0.26 | | 66 |
| Calcite | 10 | 0.001 | 0.001 | 41 |
| Kaolinite | 32 | 0.04 | 0 | 5 |

Table 5 lists some of the organic compounds identified as products during the catalytic degradation of N-3-oxoocatnoyl-DL-homoserine wherein the adsorbent/catalytic inhibitor is Copper-Calibrin® Z.

TABLE 5
The list of main organic molecules identified as products during the
catalytic degradation of N-3-oxooctanoyl-DL-homoserine.

| Code | Structure | Comment |
|---|---|---|
| (i) | 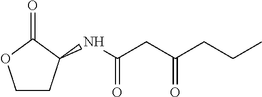 | N-3-oxooctanoyl-DL-homoserine |
| (ii) | 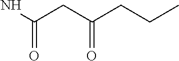 | Fragment formed by cleavage of C—N bond in N-3-oxooctanoyl-DL-homo- serine |
| (iii) | 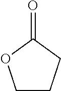 | Fragment formed by cleavage of C—N bond in N-3-oxooctanoyl-L-homoserine |
| (iv) | 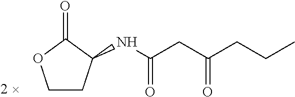 | Dimer (combination of the two original QS molecules) |
| (v) | 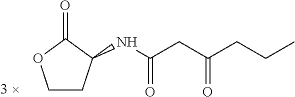 | Trimer (combination of the two original QS molecules |
| (vi) | 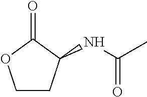 | Fragment formed by cleavage of CO—C bond in N-3-oxooctanoyl-DL-homoserine |
| (vii) | 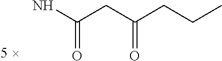 | Pentamer of one of the degraded fragments |
| (viii) | 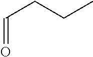 | One of the degraded fragments (a ketone) |

### Example 3 Adsorption/Catalysis of 2-heptyl-3-hydroxyl-4-quinolone (PQS) on Different Adsorbent/Catalytic Inhibitors

A 50% methanol solution containing 100 ppm 2-heptyl-3-hydroxyl-4-quinolone was taken in a small vial with a fixed amount of clay to achieve an inhibitor/analyte ratio of 100 (mg/mg). The suspension was agitated at 100 rpm for 15 minutes, and successively centrifuged at 3,500 rpm for 30 minutes. The supernatant was drawn off and directly analyzed with HPLC-DAD using the conditions listed in Table 6.

TABLE 6
HPLC-DAD methodology for quantification
of 2-heptyl-3-hydroxyl-4-quinolone.

| | |
|---|---|
| Column: | Agilent Zorbax RX-C8 column (150 mm × 4.6 mm × 5 μm) |
| Flow Rate: | 1.0 ml/min |
| DAD: | 340 nm |

| | % Methanol (1% Glacial | % Water (1% Glacial |
|---|---|---|
| Time (minutes) | Acetic Acid) | Acetic Acid) |
| 0.0 | 60 | 40 |
| 10.0 | 60 | 40 |
| 15.0 | 100 | 0 |
| 20.0 | 100 | 0 |
| 21.0 | 60 | 40 |
| 24.0 | 60 | 40 |

The performance of the different adsorbent/catalytic inhibitors for 2-heptyl-3-hydroxyl-4-quinolone is provided in Table 7.

TABLE 7
The performance of the different clays/modified materials
for the removal of pseudomonas quinolone signal (PQS)
at inhibitor/QS analyte ratio of 100.

| Material | % QS Removal |
|---|---|
| Activated Carbon | 100 |
| Aluminum Chloride | 56 |
| Calibrin ®-A | 35 |
| Calibrin ®-A-Histidine | 53 |
| Calibrin ®-A-lsoleucine | 67 |
| Calibrin ®-A-Lysozyme | 53 |
| Calibrin ®-Z | 67 |
| Calibrin ®-TQ | 72 |
| Calibrin ®-Z-Lysozyme | 45 |
| Humic Acid | 60 |
| HY-Zeolite | 98 |
| Montmorillonite - Purified | 74 |
| Montmorillonite-Al | 99 |
| Montmorillonite-Cu | 99 |
| Montmorillonite-H | 99 |

### Example 4 Adsorption/Catalysis of (S)-4,5-dihydroxy-2,3-pentanedione (AI-2) on Different Adsorbent/Catalytic Inhibitors

A 5 ppm solution (pH 3) of (S)-4,5-dihydroxy-2,3-pentanedione was taken in a small vial containing a fixed amount of clay to achieve an inhibitor/analyte ratio of 50,000 (mg/mg). The suspension was agitated at 100 rpm for 15 minutes, and successively centrifuged at 3,500 rpm for 30 minutes. The supernatant was drawn off and mixed with an equal volume of 0.1 M HCl containing 200 ppm of 2,3-diaminonaphthalene for derivatization. The solutions were heated in a 90° C. water bath for 40 minutes, and directly analyzed with HPLC-FLD using the conditions listed in Table 8.

TABLE 8
HPLC-FLD methodology for quantification
of (S)-4,5-dihydroxy-2,3-pentanedione.

| | |
|---|---|
| Column: | Phenomenex Kinetex C18 column |
| | (250 mm × 4.6 mm × 5 μm) |
| Flow Rate: | 0.8 ml/min |
| FLD: | Ex: 271 nm; Em: 503 nm |

| Time (minutes) | % Water (0.1% Formic Acid) | % Acetonitrile |
|---|---|---|
| 0.0 | 70 | 30 |
| 4.0 | 70 | 30 |
| 12.0 | 35 | 65 |
| 20.0 | 35 | 65 |
| 24.0 | 70 | 30 |
| 27.0 | 70 | 30 |

The performance of the different adsorbent/catalytic inhibitors for the removal of 2-heptyl-3-hydroxyl-4-quinolone is provided in Table 9.

TABLE 9
The performance of different clays/modified materials
for the removal of 4,5-dihydroxy-2,3-pentanedione
(AI-2) at an inhibitor/analyte ratio of 50,000.

| Material | % QS removal |
|---|---|
| Activated Carbon | 100 |
| Attapulgite | 43 |
| Attapulgite-Lysozyme | 21 |
| Calibrin ®-A-Histidine | 21 |
| Calibrin ®-A-lsoleucine | 17 |
| HY-Zeolite | 63 |
| Illite | 39 |
| Montmorillonite - Purified | 17 |
| Montmorillonite-Al | 13 |
| Montmorillonite-Cu | 33 |

The results show that the Calibrin and modified clays were able to adsorb or catalytically degrade various quorum sensing molecules at 25° C. in different media.

### Example 5 QA Disruption/Inhibition in V. harveyi by Clay-Based Adsorbent/Catalytic Inhibitors

General Method: 5 μl of a bacterial culture (V. harveyi ACTCC 14126) was inoculated in 5 mL of Luria broth (“LB”) containing 2% NaCl with different clay-base adsorbent/catalytic inhibitors (50 mg/mL, 10 mg/mL, 1 mg/mL, 100 μg/mL and 100 μg/mL). A parallel medium control experiment was also conducted without any clays.

The bacterial culture was incubated at 30° C. with agitation (200 rpm) for 9-10 h. During the culturing period, bacterial growth was monitored by measuring the optical density of the samples at 600 nm (BioPhotometer, Eppendorf) or by viable bacterial cell count on LB+ agar plates. The luminescent emission was detected and quantified by a luminescence detector (MiniLumat, EG&G Berhold) every hour.

Emission of bacterial luminescence was measured from the culture treated with different concentrations of fine Calibrin® Z. Vibrio parahaemolyticus (Vₚ) was the medium control. Total luminescence of the Area Under Curve (AUC) was calculated by measuring the luminescence between two adjacent time points for each treatment was determined by the following formula: AUC1=(L1+L2)/2*(T2−T1), in which T1 and T2 stand for time points 1 and 2, respectively, while L1 and L2 stand for the luminescence at time points 1 and 2. The total AUC was then determined by summing the AUCs for each pair of time points. Relative luminescent emission (%) of AUC is indicated by the numbers in the bars; bacterial growth was expressed by measuring optical density at 600 nm.

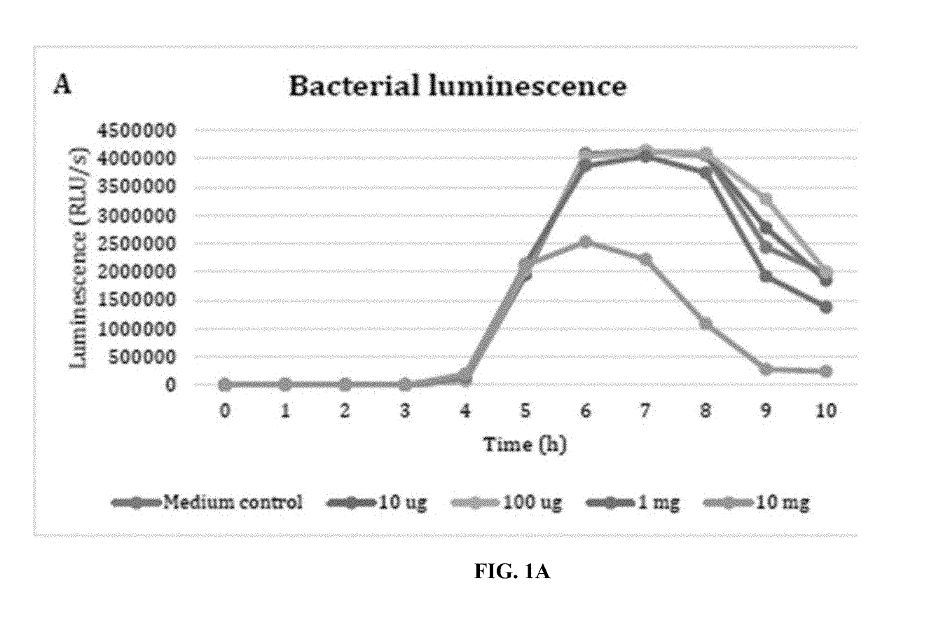

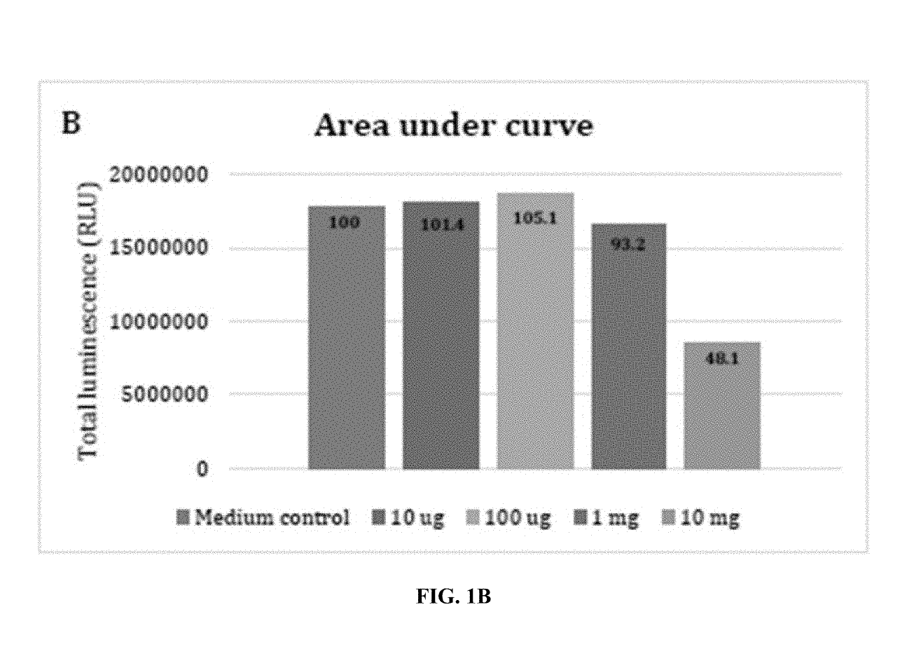

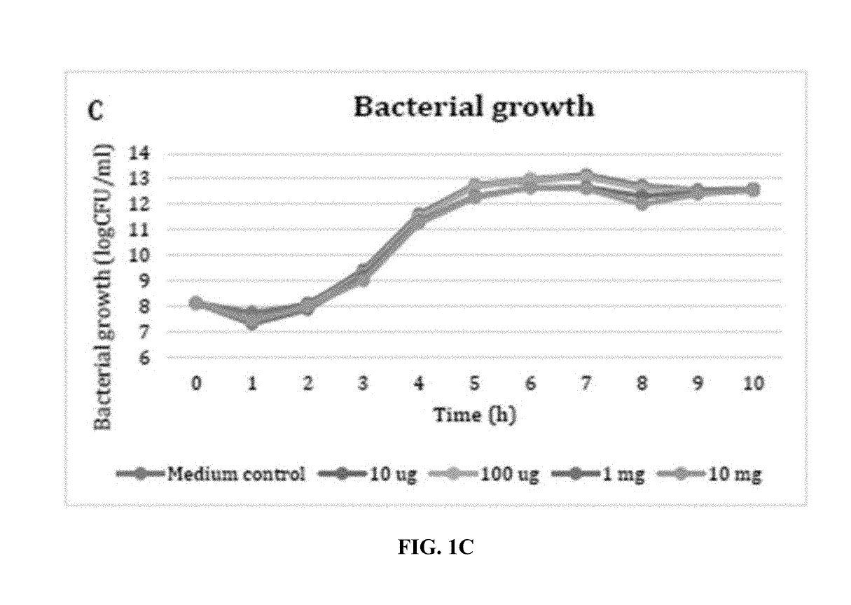

FIG. 1 shows the results obtained when Calibrin® Z was used as the adsorbent/catalytic inhibitor. As can be seen from FIGS. 1A and 1B, the bacterial luminescence produced by V. harveyi decreased as the amount of Calibrin® Z increased in the bacterial culture. The reduction of total luminescence suggests a decrease of total toxin produced in the system. The amount of adsorbent/catalytic inhibitor required to significantly bring down the luminescence was 1 mg/mL. The observation that bacterial concentration did not go down with the addition of clay suggests that the adsorbent/catalytic inhibitor selectively removed QS molecules without killing the bacteria. A significant drop in luminescence, as gauged from the area under the luminescence curve, was observed at an adsorbent/catalytic concentration of 10 mg/mL.

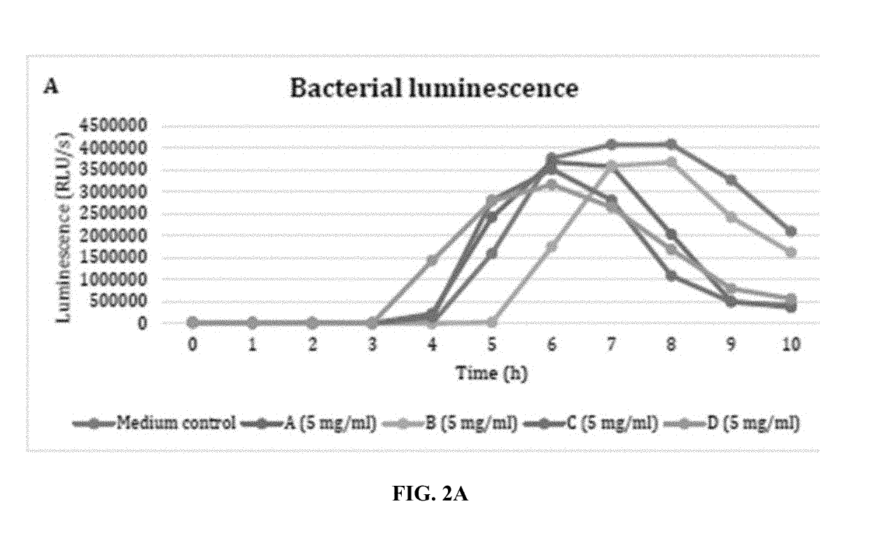

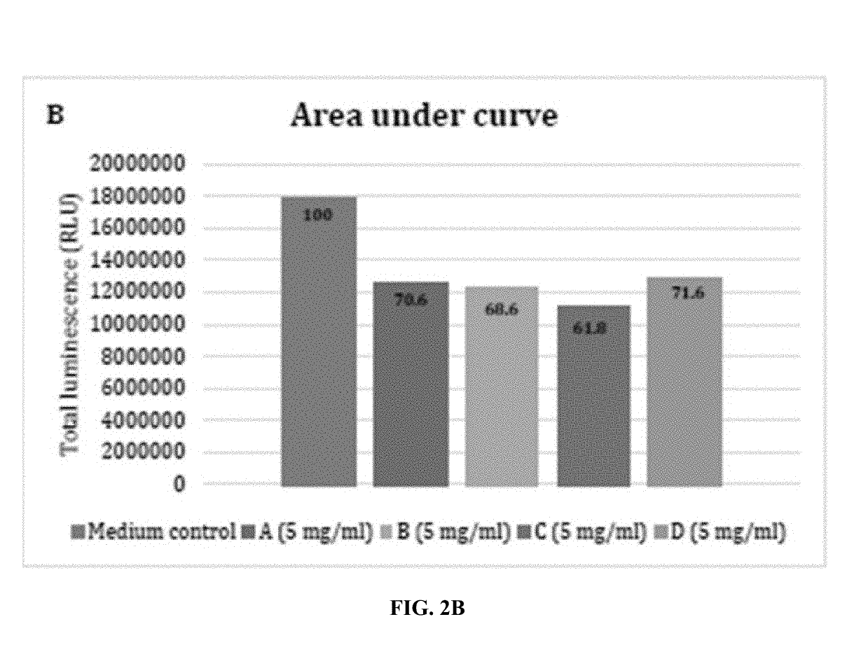

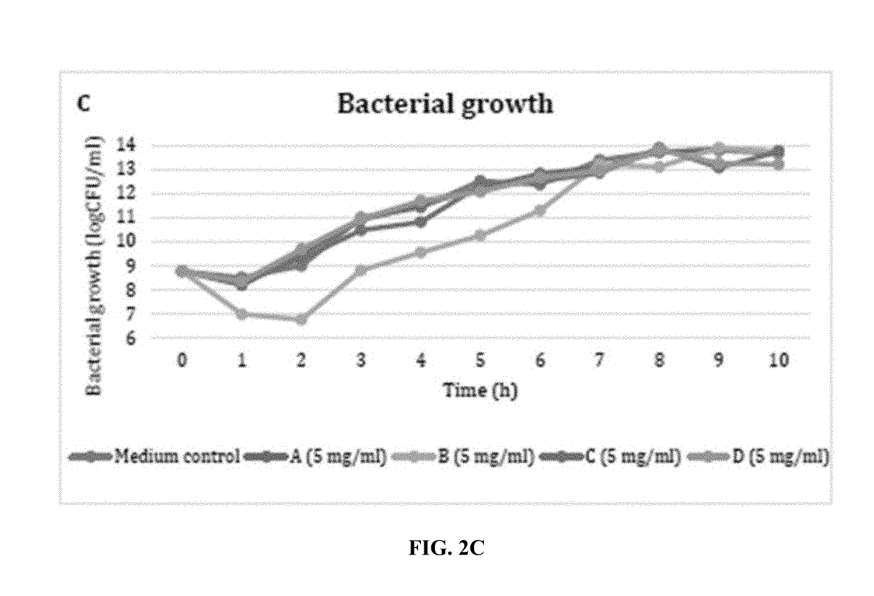

FIG. 2 illustrate the results observed when four different adsorbent/catalytic inhibitors, Calibrin® Z (A), Cu-Calibrin® Z (B), H-Calibrin® Z (C) and activated carbon (D) were used to disrupt QS in V. harveyi; the concentration of the adsorbent/catalytic inhibitor was 5 mg/mL.

A reduction in the bacterial luminescence was observed for each of the absorbent/catalytic inhibitors. These observations suggest that adsorbent/catalytic inhibitors adsorbed or broke down QS molecules in the medium. Of the four adsorbent/catalytic inhibitors tested, only Cu-Calibrin® affected the total bacterial concentration; Cu-Calibrin® Z reduced the bacterial growth during the first 5/6 h of the experiment and this suggests Cu-Calibrin® Z might exhibit some antibacterial activity.

### Example 6 QA In Vitro Disruption/Inhibition in V. harveyi by Clay-Based Adsorbent/Catalytic Inhibitors

General Method: 5 μL of an overnight bacterial culture (V. harveyi, ATCC14126) was inoculated in 5 mL of LB+ broth and incubated at 30° C. for 4 h. The bacterial mass was collected by centrifugation at 4000 g for 10 min. The supernatant was then collected and filtered through a syringe filter with a 0.45 μm pore size (PALL); 5 mL of the filtrate was then mixed with different amounts of clay based materials to reach a final QS adsorbent/inhibitor concentrations.

The treated filtrates were then incubated at 30° C. for 1 h with agitation (200 rpm). A blank filtrate containing no added clay was included as a control in each experiment. After incubation, the clay materials were eliminated from the filtrate by centrifugation at 2000×g and filtration through a syringe filter with a 0.45 μm pore size (PALL). After the removal the clay products, 4 μL of a young bacterial culture (is a culture that was not yet luminescing) was inoculated into 4 mL of each filtrate containing QS molecules, and the mixture was incubated at 30° C. The luminescent emission and the bacterial growth were quantified by a luminescence detector (MiniLumat, EG &G Berthold) and monitored by measuring the optical density at 600 nm (BioPhotometer, Eppendorf) at various time points from 0-3 h.

Relative luminescent emission (%) was measured after treatment with different concentrations of product. The amount of luminescence at each time point relative to that of the medium control is indicated by the number above the corresponding bar. Bacterial growth was measured by optical density at 600 nm.

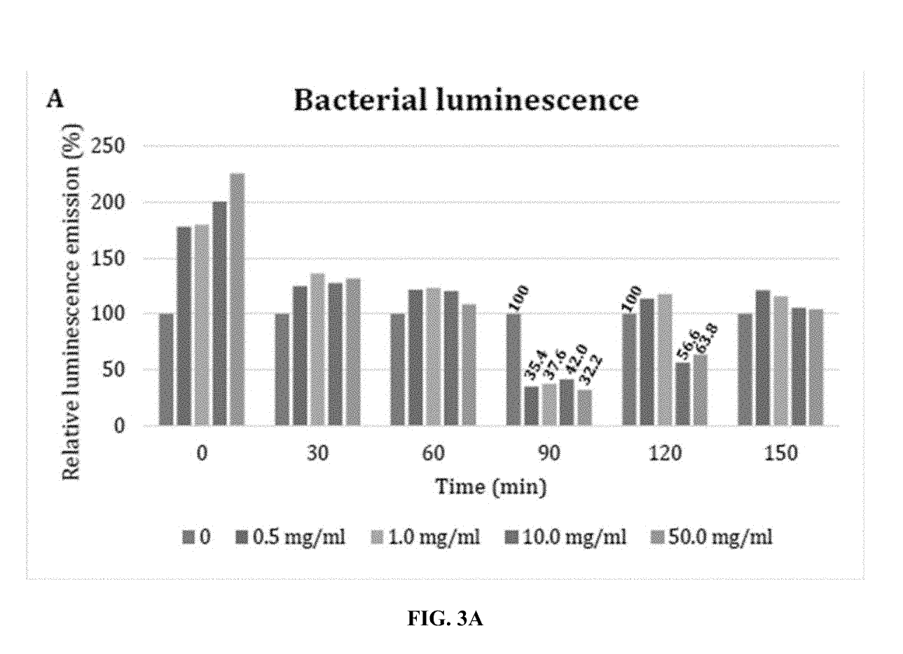

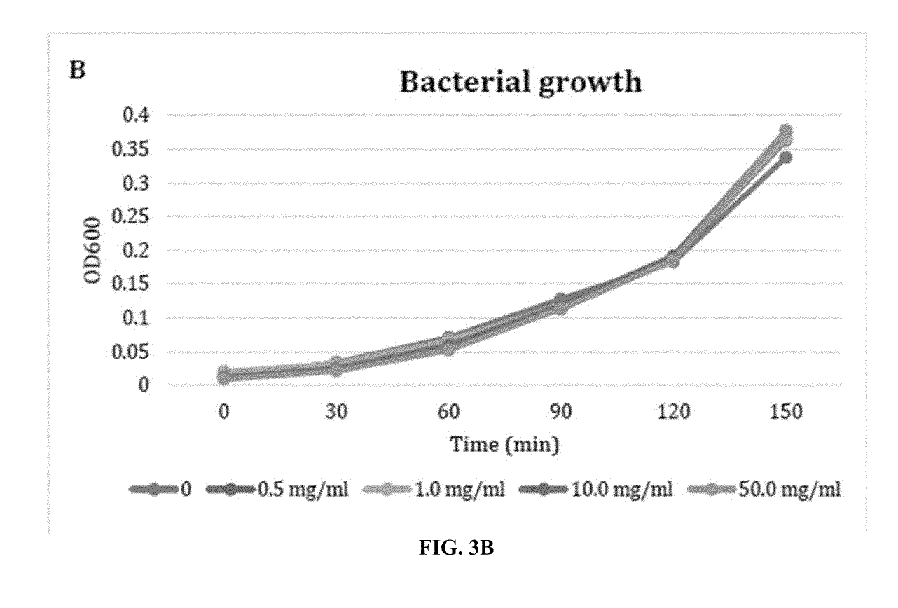

FIGS. 3A and 3B illustrate the results observed in in vitro experiments using Calibrin® Z clay to disrupt QS in V. harveyi under different conditions, where bacterial luminescence and number of bacteria were monitored over time

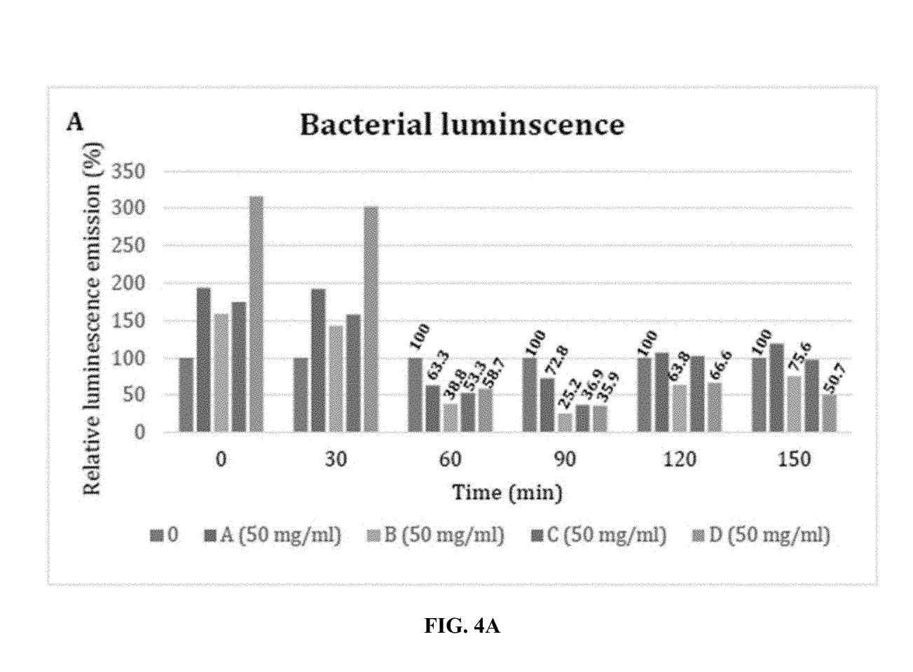

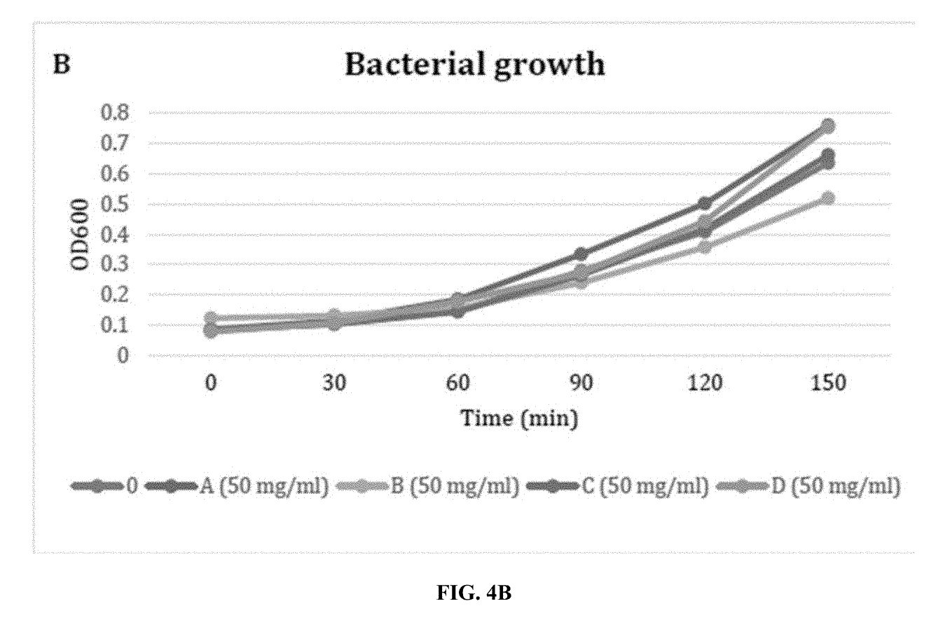

FIGS. 4A and 4B illustrate the results observed when four different adsorbent/catalytic inhibitors, Calibrin® Z (A), Cu-Calibrin® Z (B), H-Calibrin® Z (C) and activated carbon (D) were used to disrupt QS in V. harveyi in in vitro experiments under different conditions, where bacterial luminescence and number of bacteria were monitored over time. As can be seen from FIG. 4A, there is a clear delay in the onset of bacterial luminescence in the clay-treated samples after the first adsorption of QS molecules. The delay suggests that QS molecules were adsorbed by the clay and their concentration was lower than that in standard sample which was not clay treated. FIG. 4B, indicates that no difference in bacterial growth was observed in the first 60 min of incubation; however, Cu-Calibrin® Z-containing product clearly showed reduced the bacterial growth at later time points.

The invention is further described by the following numbered paragraphs:

#1. A method for modulating the flora of bacteria in an environment by inhibiting the quorum sensing of a specific bacteria in said environment, which comprises identifying the bacteria for which the quorum sensing is to be inhibited and administering an effective amount of a quorum sensing control composition comprising at least one quorum sensing control agent, which is an adsorbent/catalytic inhibitor of a QS signal sensing molecule, and optionally an inert carrier to said environment.

#2. A method for modulating the flora of bacteria in an environment by inhibiting the quorum sensing of a specific bacteria in said environment, which comprises identifying the bacteria for which the quorum sensing is to be inhibited and administering an effective amount at least one quorum sensing control agent, which is an adsorbent/catalytic inhibitor of a QS signal sensing molecule, wherein said QS signal molecule is an AHL, PQS, AI-1 signal or AI-2 signal, and optionally an inert carrier to said environment.

#3. A method for modulating the flora of bacteria in an environment by inhibiting the quorum sensing of a specific bacteria in said environment, which comprises identifying the bacteria for which the quorum sensing is to be inhibited and administering an effective amount of a quorum sensing control composition comprising at least one quorum sensing control agent, which is an adsorbent/catalytic inhibitor of a QS signal and is a clay, silica, calcite, zeolite, a sorbent mineral, diatomaceous earth, smectite, activated carbon, a nanoparticle or a combination of any of the foregoing, and optionally an inert carrier to said environment.

#4. The method according to paragraph #3, wherein the adsorbent/catalytic material is a silica, calcite, zeolite, a sorbent mineral, diatomaceous earth or activated carbon.

#5. The method according to paragraph #4, wherein the adsorbent/catalytic material is a clay.

#6. The method according to paragraph #5, wherein the clay is a silicate.

#7. The method according to paragraph #6, wherein the silicate clay is a montmorillonite clay.

#8. The method according to paragraph #6, wherein the silicate clay is a calcium montmorillonite clay.

#9. The method according to paragraph #6, wherein the silicate clay is a thermally processed montmorillonite clay.

#10. The method according to paragraph #9, wherein the thermally processed montmorillonite clay is Calibrin® A or Calibrin® Z.

#11. The method according to paragraph #5, wherein the clay is a hormite or attapulgite clay.

#12. The method according to paragraph #5, wherein the clay is a modified clay.

#13. The method according to paragraph #12, wherein the modified clay is obtained by reacting a clay with an ion-exchange material or an amino acid.

#14. The method according to paragraph #12, wherein the modified clay is obtained by reacting the clay with an ion-exchange material.

#15. The method according to paragraph #14, wherein the modified clay is copper-exchanged montmorillonite clay.

#16. The method according to paragraph #14, wherein the modified clay is aluminum, copper or proton exchanged montmorillonite clay.

#17. The method according to paragraph #16, wherein the copper-exchanged montmorillonite clay is copper exchanged Calibrin® Z.

#18. The method according to paragraph #12, wherein the modified clay is obtained by reacting the clay with an amino acid.

#19. The method according to paragraph #18, wherein the amino acid is histidine or isoleucine.

#20. The method according to paragraph #18, wherein the clay is a montmorillonite clay.

#21. The method according to any one of paragraph #1, 2 or 3, wherein the environment is in or on a human or in or on an animal.

#22. The method according to any one of paragraph #1, 2 or 3, wherein the environment is an aqueous environment.

#23. The method according to paragraph #22, wherein the environment is soil, sludge, animal bedding or waste water.

#24. The method according to paragraph #21, wherein the environment is on the skin surface of a human or animal or is the gastrointestinal track nasal passages, urinal track, vaginal tract, or gut of a human or animal.

#25. The method according to paragraph #1, 2 or 3, wherein the identified bacteria is Clostridium sp., Escherichia sp., Pseudomonas sp., Salmonella sp., Vibrio sp., or a combination thereof.

#26. The method according to any one of paragraph #1, 2 or 3, wherein the identified bacteria is C. difficile, E. coli, P. aeruginosa, S. typhimurium, V. harveyi, or a combination thereof.

#27. The method according to paragraph #5, wherein the clay is a functionalized clay.

#28. The method according to paragraph #27, wherein the functionalized clay is a lysozyme functionalized clay.

#29. The method according to any one of paragraph #1, 2 or 3 wherein the environment is a food stuff.

#30. The method according to paragraph #21, wherein the animal is a chicken, sheep, cattle, pig or a companion animal.

#31. A quorum sensing control agent which comprises an effective amount of an adsorbent/catalytic inhibitor which is a modified clay that is obtained by reacting a clay with an ion-exchange agent or an amino acid and an inert carrier

#32. The quorum sensing control agent according to paragraph #31, wherein the modified clay is obtained by reacting the clay with an ion-exchange material.

#33. The quorum sensing control agent according to paragraph #32, wherein the modified clay is copper-exchanged montmorillonite clay.

#34. The quorum sensing control agent according to paragraph #31, wherein the modified clay is an aluminum, copper or proton exchanged montmorillonite clay

#35. The quorum sensing control agent according to paragraph #34, wherein the copper-exchanged montmorillonite clay is copper exchanged Calibrin® Z.

#36. The quorum sensing control agent according to paragraph #31, wherein the modified clay is obtained by reacting the clay with an amino acid.

#37. The quorum sensing control agent according to paragraph #36, wherein the amino acid is histidine or isoleucine.

#38. The quorum sensing control agent according to paragraph #37, wherein the clay is a montmorillonite clay.

#39. A quorum sensing control agent which comprises an effective amount of an adsorbent/catalytic inhibitor which is a functionalized clay and an inert carrier.

#40. The quorum sensing control agent according to paragraph #39, wherein the functionalized clay a lysozyme functionalized clay.

#41. A method for inhibiting the spoilage of foodstuffs by inhibiting the quorum sensing of a specific bacteria in said foodstuffs by administering an effective amount of a quorum sensing control composition comprising at least one quorum sensing control agent, which is an adsorbent/catalytic inhibitor a QS signal sensing molecule, and optionally an inert carrier to said foodstuffs.

#42. A method for preventing or treating vibriosis in fish or shellfish in need thereof by inhibiting the quorum sensing of Vibrio sp. in an aqueous environment where said fish or shellfish reside by administering an effective amount of a quorum sensing control composition comprising at least one quorum sensing control agent, which is an adsorbent/catalytic inhibitor a QS signal sensing molecule, and optionally an inert carrier to said aqueous environment.

#43. The method according to paragraph #42, wherein the adsorbent/catalytic material is a silica, calcite, zeolite, a sorbent mineral, diatomaceous earth or activated carbon.

#44. The method according to paragraph #42, wherein the adsorbent/catalytic material is a clay.

#45. The method according to paragraph #44, wherein the clay is a silicate.

#46. The method according to paragraph #45, wherein the silicate clay is a montmorillonite clay.

#47. The method according to paragraph #45, wherein the silicate clay is a calcium montmorillonite clay.

#48. The method according to paragraph #45, wherein the silicate clay is a thermally processed montmorillonite clay.

#49. The method according to claim 48, wherein the thermally processed montmorillonite clay is Calibrin® A or Calibrin® Z.

#50. The method according to paragraph #44, wherein the clay is a hormite or attapulgite clay.

#51. The method according to paragraph #45, wherein the silicate clay is H-Calibrin®Z.

#52. The method according to paragraph #44, wherein the clay is a modified clay.

#53. The method according to paragraph #52, wherein the modified clay is obtained by reacting a clay with an ion-exchange material or an amino acid.

#54. The method according to paragraph #52, wherein the modified clay is obtained by reacting the clay with an ion-exchange material.

#55. The method according to paragraph #53, wherein the modified clay is copper-exchanged montmorillonite clay.

#56. The method according to paragraph #53, wherein the modified clay is aluminum, copper or proton exchanged montmorillonite clay

#57. The method according to paragraph #56, wherein the copper-exchanged montmorillonite clay is copper exchanged Calibrin® Z.

#58. The method according to paragraph #52, wherein the modified clay is obtained by reacting the clay with an amino acid.

#59. The method according to paragraph #58, wherein the amino acid is histidine or isoleucine.

#60. The method according to paragraph #59, wherein the claim is a montmorillonite clay.

Having thus described in detail, preferred embodiments of the present invention, it is to be understood that the invention defined by the above paragraphs is not to be limited to particular details set forth in the above description as many apparent variations thereof are possible without departing from the spirit or scope of the present invention.

1. A method for modulating the flora of bacteria in an environment by inhibiting the quorum sensing of a specific bacteria in said environment, which comprises identifying the bacteria for which the quorum sensing is to be inhibited and administering an effective amount of a quorum sensing control composition comprising at least one quorum sensing control agent, which is an adsorbent/catalytic inhibitor of a QS signal sensing molecule, and optionally an inert carrier to said environment.

2. A method for modulating the flora of bacteria in an environment by inhibiting the quorum sensing of a specific bacteria in said environment, which comprises identifying the bacteria for which the quorum sensing is to be inhibited and administering an effective amount at least one quorum sensing control agent, which is an adsorbent/catalytic inhibitor of a QS signal sensing molecule, wherein said QS signal molecule is an AHL, PQS, AI-1 signal or AI-2 signal, and optionally an inert carrier to said environment.

3. A method for modulating the flora of bacteria in an environment by inhibiting the quorum sensing of a specific bacteria in said environment, which comprises identifying the bacteria for which the quorum sensing is to be inhibited and administering an effective amount of a quorum sensing control composition comprising at least one quorum sensing control agent, which is an adsorbent/catalytic inhibitor of a QS signal, which is an inorganic or organic sorptive material, a sorptive mineral, a sorbent mineral, or a non-porous mineral, and optionally an inert carrier to said environment.

4. The method according to claim 3, wherein the adsorbent/catalytic inhibitor is a sorptive mineral, which is a processed clay.

5. The method according to claim 3, wherein the adsorbent/catalytic inhibitor is a sorbent mineral and the sorbent mineral is a zeolite, silica, calcite, illite, volcanic silica, mica or perlite.

6. The method according to claim 4, wherein the processed clay is a heat treated clay, ion-exchanged clay, or a functionalized clay.

7. The method according to claim 6, wherein the processed clay is a heat treated clay and the clay material comprises a clay mineral, phyllosilicate, alumino-silicate, attapulgite, bentonite, hormite, or fuller's earth.

8. The method according to claim 7, wherein the clay material is montmorillonite clay.

9. The method according to claim 6, wherein the processed clay is an ion-exchanged clay and the clay material comprises a clay mineral, phyllosilicate, alumino-silicate, attapulgite, bentonite, hormite, or fuller's earth.

10. The method according to claim 9, wherein the ion-exchanged clay is aluminum, copper or proton exchanged clay.

11. The method according to claim 6, wherein the processed clay is a functionalized clay and the clay material comprises a clay mineral, phyllosilicate, alumino-silicate, attapulgite, bentonite, hormite, or fuller's earth.

12. The method according to claim 11, wherein the functionalized clay is an amino acid functionalized clay and the amino acid is histidine or isoleucine.

13. The method according to claim 11, wherein the functionalized clay is a protein functionalized clay and the protein is lysozyme.

14. The method according to claim 4, wherein the adsorbent/catalytic inhibitor of a QS signal is an inorganic or organic sorptive material.

15. The method according to claim 3, wherein the adsorbent/catalytic inhibitor of a QS signal is a non-porous mineral and the non-porous mineral is an aluminum oxide, a silica oxide, an iron oxide, AlCl₃, a copper oxide or calcium oxides.

16. The method according to claim 1, wherein the environment is in or on a human or in or on an animal.

17. The method according to claim 1, wherein the environment is an aqueous environment.

18. The method according to claim 1, wherein the environment is soil, sludge, animal bedding or waste water.

19. The method according to claim 1, wherein the environment is on the skin surface of a human or animal or is the gastrointestinal tract, nasal passages, urinal tract, vaginal tract, or gut of a human or animal.

20. The method according to claim 1, wherein the identified bacteria is Clostridium sp., Escherichia sp., Pseudomonas sp., Salmonella sp., Vibrio sp., or a combination thereof.

21. The method according to claim 1, wherein the identified bacteria is C. difficile, E. coli, P. aeruginosa, S. typhimurium, V. harveyi, or a combination thereof.

22. The method according to claim 1, wherein the environment is a food stuff.

23. The method according to claim 19, wherein the animal is poultry, sheep, cattle, horse, pig or a companion animal.

24. A quorum sensing control agent which comprises an effective amount adsorbent/catalytic inhibitor of a QS signal is an inorganic or organic sorptive material, sorptive mineral, non-porous mineral or a combination of any of the foregoing and an inert carrier.

25. A method for inhibiting the spoilage of foodstuffs by inhibiting the quorum sensing of a specific bacteria in said foodstuffs by administering an effective amount of a quorum sensing control composition comprising at least one quorum sensing control agent, which comprises an adsorbent/catalytic inhibitor a QS signal sensing molecule and optionally an inert carrier, to said foodstuffs.

26. A method for preventing or treating vibriosis in fish or shellfish in need thereof by inhibiting the quorum sensing of Vibrio sp. in an aqueous environment where said fish or shellfish reside by administering an effective amount of a quorum sensing control composition comprising at least one quorum sensing control agent, which is an adsorbent/catalytic inhibitor a QS signal sensing molecule, and optionally an inert carrier to said aqueous environment.

27. The method according to claim 26, wherein the adsorbent/catalytic material is an inorganic or organic sorptive material, sorptive mineral, a sorbent mineral, or a non-porous mineral or a mixture of the foregoing.

28. A method of eliminating or reducing production of a biofilm or toxic chemicals produced by targeted genus or species of bacteria residing in an animal or human by eliminating or reducing a QS signal molecule produced by said bacteria, which comprises administering an effective amount of a quorum sensing control composition comprising at least one quorum control agent, which is an adsorbent/catalytic inhibitor for the QS signal molecule and optionally an inert carrier.

29. The method according to claim 28, wherein the QS signal molecule is an AHL, a PQS, AI-1 signal, or an AI-2 signal.

30. The method according to claim 28, wherein the identified bacteria are C. difficile, E. coli, P. aeruginosa, S. typhimurium, V. harveyi, or a combination thereof.

31. The method according to claim 28, wherein the adsorbent/catalytic material is an organic or inorganic sorptive material, sorptive mineral, a sorbent mineral, or a non-porous mineral or a mixture of the foregoing.

32. A method for eliminating or chemically inactivating at least one QS signal molecule produced by targeted bacteria which comprises administering an effective amount of a quorum sensing control composition comprising at least one quorum sensing control agent, which is an adsorbent/catalytic inhibitor for the QS signal molecule, and optionally an inert carrier, to an environment where said targeted bacteria reside.

33. The method according to claim 32, wherein the QS signal molecule is an AHL, a PQS, AI-1 signal, or an AI-2 signal.

34. The method according to claim 32, wherein the identified bacteria are C. difficile, E. coli, P. aeruginosa, S. typhimurium, V. harveyi, or a combination thereof.

35. The method according to claim 32, wherein the adsorbent/catalytic material is an inorganic or organic sorptive material, sorptive mineral, a sorbent mineral, or a non-porous mineral or a mixture of the foregoing.
